(19) United States
(12) Patent Application Publication
(10) Pub. No.: US 2014/0335084 A1
(43) Pub. Date: Nov. 13, 2014

(54) ANTIBODY FORMULATION

(71) Applicant: Hoffmann-La Roche Inc., Little Falls, NJ (US)

(72) Inventors: Jonas Fast, Riehen (CH); Stefan Fischer, Basel (CH); Robert Mueller, Basel (CH)

(73) Assignee: Hoffmann-La Roche Inc., Little Falls, NJ (US)

(21) Appl. No.: 14/363,103

(22) PCT Filed: Dec. 3, 2012
(86) PCT No.: PCT/EP2012/074182

§ 371 Date: Jun. 5, 2014

(30) Foreign Application Priority Data
Dec. 6, 2011 (EP) 11192243.1

(51) Int. Cl. C07K 16/28

(52) U.S. Cl. CPC C07K 16/2884; C07K 2317/24
USPC 424/133.1; 424/172.1

(57) ABSTRACT

The present invention relates to an anti-CD44 monoclonal antibody formulation, a process for the preparation of said formulation and uses thereof.

### FIELD OF THE INVENTION

The present invention relates to a humanized anti-CD44 monoclonal antibody formulation, a process for the preparation of said formulation and uses thereof.

### BACKGROUND OF THE INVENTION

CD44 in Cancer: Raising monoclonal antibodies against human white blood cells led to the discovery of the CD44 antigen; a single chain hyaluronic acid (HA) binding glycoprotein expressed on a wide variety of normal tissue and on all types of hematopoietic cells. It was originally associated with lymphocyte activation and homing. Currently, its putative physiological role also includes activation of inflammatory genes, modulation of cell cycle, induction of cell proliferation, induction of differentiation and development, induction of cytoskeletal reorganization and cell migration and cell survival/resistance to apoptosis.

In humans, the single gene copy of CD44 is located on the short arm of chromosome 11, 11p13. The gene contains 19 exons; the first 5 are constant, the next 9 are variant, the following 3 are constant and the final 2 are variant. Differential splicing can lead to over 1000 different isoforms. However, currently only several dozen naturally occurring variants have been identified.

The CD44 standard glycoprotein consists of a N-terminal extracellular (including a 20 a.a. leader sequence, and a membrane proximal region (85 a.a.)) domain (270 a.a.), a transmembrane region (21 a.a.) and a cytoplasmic tail (72 a.a.). The extracellular region also contains a link module at the N-terminus. This region is 92 a.a. in length and shows homology to other HA binding link proteins. There is high homology between the mouse and human forms of CD44. The variant forms of the protein are inserted to the carboxy terminus of exon 5 and are located extracellularly when expressed.

A serum soluble form of CD44 also occurs naturally and can arise from either a stop codon (within the variable region) or from proteolytic activity. Activation of cells from a variety of stimuli including TNF-α results in shedding of the CD44 receptor. Shedding of the receptor has also been seen with tumor cells and can result in an increase in the human serum concentration of CD44 by up to 10-fold. High CD44 serum concentration suggests malignancy (ovarian cancer being the exception).

The standard form of CD44 exists with a molecular weight of approximately 37 kD. Post-translational modifications increase the molecular weight to 80-90 kD. These modifications include amino terminus extracellular domain N-linked glycosylations at asparagine residues, O-linked glycosylations at serine/threonine residues at the carboxy terminus of the extracellular domain and glycosaminoglycan additions. Splice variants can range in size from 80-250 kD.

HA, a polysaccharide located on the extracellular matrix (ECM) in mammals, is thought to be the primary CD44 ligand. However, CD44 has also been found to bind such proteins as collagen, fibronectin, laminin etc. There appears to be a correlation between HA binding and glycosylation. Inactive CD44 (does not bind HA) has the highest levels of glycosylation, active CD44 (binding HA) the lowest while inducible CD44 (does not or weakly binds HA unless activated by cytokines, monoclonal antibodies, growth factors, etc.) has glycoslyation levels somewhere in between the active and inactive forms.

CD44 can mediate some of its functions through signal transduction pathways that depend on the interaction of the cell, stimulus and the environment. Some of these pathways include the NFκB signaling cascade (involved in the inflammatory response), the Ras-MAPK signal transduction pathway (involved with activating cell cycling and proliferation), the Rho family of proteins (involved with cytoskeleton reorganization and cell migration) and the PI3-K-related signaling pathway (related to cell survival). All of the above-mentioned functions are closely associated with tumor disease initiation and progression. CD44 has also been implicated in playing a role in cancer through a variety of additional mechanisms. These include the presentation of growth factors, chemokines and cytokines by cell surface proteoglycans present on the cell surface of CD44 to receptors involved in malignancy. Also, the intracellular degradation of HA by lysosomal hyaluronidases after internalization of the CD44-HA complex can potentially increase the likelihood of tumor invasiveness and induction of angiogenesis through the ECM. In addition, the transmission of survival or apoptotic signals has been shown to occur through either the standard or variable CD44 receptor. CD44 has also been suggested to be involved in cell differentiation and migration. Many, if not all, of these mechanisms are environment and cell dependent and several give rise to variable findings. Therefore, more research is required before any conclusions can be drawn.

In order to validate a potential functional role of CD44 in cancer, expression studies of CD44 were undertaken to determine if differential expression of the receptor correlates with disease progression. However, inconsistent findings were observed in a majority of tumor types and this is probably due to a combination of reagents, technique, pathological scoring and cell type differences between researchers. Renal cell carcinoma and non-Hodgkin's lymphoma appear to be the exception in that patients with high CD44 expressing tumors consistently had shorter survival times than their low or non-CD44 expressing counterparts.

Due to its association with cancer, CD44 has been the target of the development of anti-cancer therapeutics. There is still controversy as to whether the standard or the variant forms of CD44 are required for tumor progression. There is in vivo animal data to support both views and again it may be tumor type and even cell type dependent. Different therapeutic approaches have included injection of soluble CD44 proteins, hyaluronan synthase cDNA, hyaluronidase, the use of CD44 antisense and CD44 specific antibodies. Each approach has led to some degree of success thereby providing support for anti-CD44 cancer therapeutics.

Both variant and standard CD44 specific monoclonal antibodies have been generated experimentally but for the most part these antibodies have no intrinsic biological activity, rather they bind specifically to the type of CD44 they recognize.

VFF-18, also designated as BIWA1, is a high-affinity antibody to the v6 variant of CD44, specific for the 360-370 region of the polypeptide. Various versions or derivatives of BIWA1 have undergone clinical testing for the treatment of cancer. In particular, BIWA1 was been used as a ^{99m}Technetium-labelled conjugate in a Phase 1 clinical trial for the testing of safety and targeting potential in 12 patients with squamous cell carcinoma of the head and neck. Forty hours after injection, 14 percent of the injected dose was taken up by the tumor, with minimal accumulation in other organs such as the kidney, spleen and bone marrow. Although the highly selective tumor binding suggests a role for BIWA1 in radioimmunotherapy, the exceptionally high affinity of this antibody prevented penetration into the deeper layers of the tumor. Further, BIWA1 was also found to be significantly immunogenic. Eleven of the twelve study patients developed human anti-mouse antibodies (HAMA) that exhibited heterogenous accumulation throughout the tumor and led to formation of antibody-soluble CD44 complexes.

WO 02/094879 discloses humanized versions of VFF-18 designed to overcome the HAMA response, designated BIWA4 and BIWA8. BIWA4 was found to have a significantly lower antigen binding affinity compared to the parent VFF 18 antibody. However, the lower affinity BIWA4 antibody demonstrated superior tumor uptake characteristics relative to the higher affinity BIWA8. Both ^{99m}Technetium-labelled and ¹⁸⁶Rhenium-labelled BIWA4 antibodies were assessed in a 33 patient Phase 1 clinical trial to determine safety, tolerability, tumor accumulation and, for ¹⁸⁶Re-labelled BIWA4, maximum tolerated dose. There appeared to be tumor related uptake of ^{99m}Tc-labelled BIWA4. There were no tumor responses observed at any dose of ¹⁸⁶Re-labelled BIWA4. Although a number of patients demonstrated stable disease throughout the course of the study; the dose limiting toxicity occurred at 60 mCi/m². There was also a 50-65 percent rate of adverse events with 12 of 33 patients deemed to have serious adverse events (thrombocytopenia, leucopenia and fever). Additionally, 6 of these 12 patients, all treated with ¹⁸⁶Re-labelled BIWA4, died in the course of treatment or follow-up due to disease progression. Two patients also developed human anti-human antibodies (HAHA). Accordingly, the radio-labeled VFF-18 failed to demonstrate any clinical effects.

U.S. Patent Application US 2003/0103985 discloses a humanized version of VFF-18 (BIWA 4) conjugated to a maytansinoid for use in tumor therapy, designated BIWI 1. BIWI 1 was found to have significant anti-tumor effects in mouse models of human epidermoid carcinoma of the vulva, squamous cell carcinoma of the pharynx and breast carcinoma. The unconjugated version, i.e., BIWA 4, did not exhibit anti-tumor effects. The conjugate BIWI1 has no evidence of safety or efficacy in humans.

Mab U36 is a murine monoclonal IgG1 antibody developed by immunization with UM-SCC-22B human hypopharyngeal carcinoma cells and was selected for cancer and tissue specificity. Antigen characterization through cDNA cloning and sequence analysis identified the epitope of Mab U36 to be within the v6 domain of keratinocyte-specific CD44 splice variant epican. Immunohistochemistry studies show the recognized epitope to be restricted to the cell membrane. Mab U36 labeled 94 percent of the head and neck squamous cell carcinomas (HNSCC) strongly, and within these tumors there was uniformity in cell staining.

A 10 patient ^{99m}Tc-labelled Mab U36 study showed selective accumulation of the antibody to HNSCC cancers (20.4+/−12.4 percent injected dose/kg at 2 days); no adverse effects were reported but two patients developed HAMA. In a study of radio-iodinated murine Mab U36, there were 3 cases of HAMA in 18 patients and selective homogenous uptake in HNSCC. In order to decrease the antigenicity of Mab U36 and decrease the rate of HAMA, a chimeric antibody was constructed, with neither the chimeric nor the original murine Mab U36 exhibiting ADCC activity. However, the study found no evidence of anti-cancer activity of unlabeled Mab U36.

¹⁸⁶Re-labelled chimeric Mab U36 was used to determine the utility of Mab U36 as a therapeutic agent. In this Phase 1 escalating dose trial, 13 patients received a scouting dose of ^{99m}Tc-labelled chimeric Mab U36 followed by ¹⁸⁶Re-labelled chimeric Mab U36. No acute adverse events were reported. However, following treatment, dose limiting myelotoxcity (1.5 GBq/m²) was noted in 2 of 3 patients, and thrombocytopenia was observed in one patient treated with the maximum tolerated dose (1.0 GBq/m²). Although there were some effects on tumor size, these effects did not fulfill the criteria for objective responses to treatment. A further study of ¹⁸⁶Re-labelled chimeric Mab U36 employed a strategy of using granulocyte colony-stimulating factor stimulated whole blood reinfusion to double the maximum-tolerated activity to 2.8 Gy. In this study of nine patients with various tumors of the head and neck, 3 required transfusions for drug related anemia. Other toxicity included grade 3 myelotoxicity and grade 2 mucositis. No objective tumor responses were reported, although stable disease was achieved for 3-5 months in 5 patients.

### SUMMARY OF THE INVENTION

In one aspect, the invention relates to a CD44 antibody formulation comprising:
about 5 to about 30 mg/mL of a CD44 antibody,
about 5 to about 50 mM of a buffer,
at a pH from about 4.5 to about 7.0.

In another aspect, the invention relates to a CD44 antibody formulation comprising:
about 5 to about 30 mg/mL of a CD44 antibody,
about 10 to about 30 mM of a buffer,
at a pH from about 5.0 to about 6.5.

In another aspect, the invention relates to a CD44 antibody formulation further comprising about 0.005% to about 0.06% of at least one surfactant.

In another aspect, the invention relates to a CD44 antibody formulation further comprising at least one stabilizer.

In another aspect, the invention relates to a CD44 antibody formulation comprising:
about 5 to about 30 mg/mL of a CD44 antibody,
at least one further stabilizer, and
about 0.02 to about 0.04% of at least one surfactant, and
about 15 to about 25 mM of a buffer,
at a pH from about 5.0 to about 6.0.

In another preferred aspect, the stabilizers are selected from the group of sugars and amino acids.

In another aspect, the invention relates to a CD44 antibody formulation, wherein the formulation comprises:
about 5 to about 30 mg/mL of a of a CD44 antibody,
about 10 to about 30 mM of a buffer,
about 150 mM to about 300 mM sucrose
at a pH in the range from about 5.0 to about 6.5.

In another aspect, the invention relates to a CD44 antibody formulation, wherein the formulation comprises:
about 5 to about 30 mg/mL of a of a CD44 antibody,
about 10 to about 30 mM of a buffer,
about 100 mM to about 200 mM NaCl,
at a pH in the range from about 5.0 to about 6.5.

In another preferred aspect, the stabilizer is trehalose.

In another aspect, the invention relates to a CD44 antibody formulation, wherein the formulation comprises about 200 mM to about 300 mM trehalose.

In another aspect, the invention relates to a CD44 antibody formulation, wherein the formulation comprises:
about 5 to about 30 mg/mL of a of a CD44 antibody,
about 10 to about 30 mM of a buffer,
about 200 mM to about 300 mM trehalose
at a pH in the range from about 5.0 to about 6.5.

In another aspect, the invention relates to a CD44 antibody formulation, wherein the formulation comprises:
about 5 to about 30 mg/mL of a of a CD44 antibody,
about 10 to about 30 mM of a buffer,
about 200 mM to about 300 mM trehalose
5 mM to about 50 mM amino acid
at a pH in the range from about 5.0 to about 6.5.

In another preferred aspect, the invention relates to a CD44 antibody formulation, wherein the stabilizer is methionine.

In another preferred aspect, the invention relates to a CD44 antibody formulation, wherein the formulation comprises about 5 mM to about 30 mM methionine.

In another preferred aspect, the invention relates to a CD44 antibody formulation, wherein the formulation comprises trehalose and methionine.

In another preferred aspect, the invention relates to a CD44 antibody formulation, wherein the formulation comprises about 230 mM to 250 mM trehalose and about 5 mM to 15 mM methionine.

In another preferred aspect, the invention relates to a CD44 antibody formulation comprising:
about 5 to about 30 mg/mL of a CD44 antibody,
at least one further stabilizer,
about 0.02 to about 0.04% of at least one surfactant,
about 15 to about 25 mM of a buffer,
at a pH from about 5.0 to about 6.0.

In another preferred aspect, the invention relates to a CD44 antibody formulation, wherein the formulation comprises about 240 mM trehalose and about 10 mM methionine.

In another preferred aspect, the invention relates to a CD44 antibody formulation, comprising about 10 mM methionine.

In another aspect, the invention relates to a CD44 antibody formulation, which is in a liquid form, in a lyophilized form or in a liquid form reconstituted from a lyophilized form.

In another aspect, the invention relates to a CD44 antibody formulation, which can be administered by intravenous (i.v.) or subcutaneous (s.c.) or any other parental administration.

In another preferred aspect, the invention relates to a liquid CD44 antibody formulation, wherein it comprises:
about 10 mg/mL of a CD44 antibody,
about 20 mM L-histidine,
about 150 mM NaCl,
about 0.01% polysorbate 80,

at pH of about 6.0;

or
about 10 mg/mL of a CD44 antibody,
about 20 mM L-histidine,
about 240 mM trehalose,
about 0.02% polysorbate 20,

at a pH of about 6.0;

or
about 10 mg/mL of a CD44 antibody,
about 20 mM citrate,
about 190 mM sucrose,
about 20 mM L-arginine,
about 0.02% polysorbate 20,

at a pH of about 5.5;

or
about 25 mg/mL of a CD44 antibody,
about 20 mM L-histidine,
about 140 mM NaCl,
about 0.02% Poloxamer 188,

at a pH of about 5.5;

or
about 25 mg/mL of a CD44 antibody,
about 20 mM L-histidine,
about 140 mM NaCl,
about 0.02% Polysorbate 20,

at a pH of about 5.5;

or
about 25 mg/mL of a CD44 antibody,
about 20 mM L-histidine,
about 140 mM NaCl,
about 0.02% Polysorbate 20,

at a pH of about 6.0;

or
about 25 mg/mL of a CD44 antibody,
about 20 mM L-histidine,
about 140 mM NaCl,
about 0.02% Poloxamer 188,

at a pH of about 6.0;

or
about 25 mg/mL of a CD44 antibody,
about 20 mM L-histidine,
about 240 mM Sucrose,
about 0.02% Poloxamer 188,

at a pH of about 5.5;

or
about 25 mg/mL of a CD44 antibody,
about 20 mM L-histidine,
about 240 mM Sucrose,
about 0.02% Poloxamer 188,

at a pH of about 6.0.

In another preferred aspect, the invention relates to a lyophilized formulation, wherein it comprises:
about 10 mg/mL of a CD44 antibody,
about 20 mM L-histidine,
about 240 mM trehalose,
about 0.02% polysorbate 20,

at a pH of about 6.0;

or
about 10 mg/mL of a CD44 antibody,
about 20 mM L-histidine,
about 190 mM sucrose,
20 mM L-arginine,
about 0.02% polysorbate 20,

at a pH of about 5.5.

In another preferred aspect, the invention relates to a liquid CD44 antibody formulation, wherein it comprises:
about 10 mg/mL of a CD44 antibody,
about 150 mM NaCl,
about 20 mM of a L-histidine,
about 0.01% of polysorbate 80,

at a pH of about 6.

In another preferred aspect, the invention relates to a liquid CD44 antibody formulation, wherein it comprises:
about 25 mg/mL of a CD44 antibody,
about 20 mM L-histidine,
about 140 mM NaCl,
about 0.02% Poloxamer 188,

at a pH of about 5.5. In another preferred aspect, the invention relates to a liquid CD44 antibody formulation, wherein it comprises:
10 mg/mL of a CD44 antibody,
20 mM L-histidine,
150 mM NaCl,
0.01% polysorbate 80,

at a pH of 6.0;

or
10 mg/mL of a CD44 antibody,
20 mM L-histidine,
240 mM trehalose,
0.02% polysorbate 20,

at a pH of 6.0;

or
10 mg/mL of a CD44 antibody,
20 mM citrate,
190 mM sucrose,
20 mM L-arginine,
0.02% polysorbate 20,

at pH 5.5;

or
25 mg/mL of a CD44 antibody,
20 mM L-histidine,
140 mM NaCl,
0.02% Poloxamer 188,

at pH of 5.5;

or
25 mg/mL of a CD44 antibody,
20 mM L-histidine,
140 mM NaCl,
0.02% Polysorbate 20,

at a pH of 5.5;

or
25 mg/mL of a CD44 antibody,
20 mM L-histidine,
140 mM NaCl,
0.02% Polysorbate 20,

at a pH of 6.0;

or
25 mg/mL of a CD44 antibody,
20 mM L-histidine,
140 mM NaCl,
0.02% Poloxamer 188,

at a pH of 6.0;

or
25 mg/mL of a CD44 antibody,
20 mM L-histidine,
240 mM Sucrose,
0.02% Poloxamer 188,

at a pH of 5.5;

or
25 mg/mL of a CD44 antibody,
20 mM L-histidine,
240 mM Sucrose,
0.02% Poloxamer 188,

at a pH of 6.0.

In another preferred aspect, the invention relates to a lyophilized formulation, wherein it comprises:
10 mg/mL of a CD44 antibody,
20 mM L-histidine,
240 mM trehalose,
0.02% polysorbate 20,

at pH 6.0

or
10 mg/mL of a CD44 antibody,
20 mM L-histidine,
190 mM sucrose,
20 mM L-arginine,
0.02% polysorbate 20,

at a pH of 5.5.

In another preferred aspect, the invention relates to a CD44 antibody formulation, comprising:
10 mg/mL of a CD44 antibody,
150 mM NaCl,
20 mM of a L-histidine,
0.01% of polysorbate 80,
at a pH of 6.

In another preferred aspect, the invention relates to a CD44 antibody formulation, comprising:
25 mg/mL of a CD44 antibody,
20 mM L-histidine,
140 mM NaCl,
0.02% Poloxamer 188,
at a pH of 5.5.

In another preferred aspect, the invention relates to a CD44 antibody formulation, wherein the CD44 antibody comprises one or more of the CDRs selected from the group of SEQ ID NO:3, SEQ ID NO:4, SEQ ID NO:5, SEQ ID NO:6, SEQ ID NO:7, or SEQ ID NO:8.

In another preferred aspect, the invention relates to a CD44 antibody formulation, wherein the antibody is humanized.

In another preferred aspect, the invention relates to a CD44 antibody formulation, wherein the antibody comprises SEQ ID NO:9.

In another preferred aspect, the invention relates to a CD44 antibody formulation, wherein the antibody comprises SEQ ID NO:10.

In another preferred aspect, the invention relates to a CD44 antibody formulation, wherein the antibody further comprises SEQ ID NO:11.

In another preferred aspect, the invention relates to a CD44 antibody formulation, wherein it is:
10 mg/mL of a CD44 antibody,
0.01% polysorbate 80,
150 mM NaCl,
10 mM methionine,
20 mM L-histidine,

at pH 6.0;

or
10 mg/mL of a CD44 antibody,
0.02% polysorbate 20,
240 mM trehalose,
20 mM L-histidine,

at pH 6.0;

or
10 mg/mL of a CD44 antibody,
20 mM citrate,
0.02% polysorbate 20,
190 mM sucrose,
20 mM L-arginine,

at pH 5.5;

or
25 mg/mL of a CD44 antibody,
20 mM L-Histidine
140 mM NaCl
0.02% Poloxamer 188

at pH 5.5.

In another preferred aspect, the invention relates to a CD44 antibody formulation, wherein it is:
10 mg/mL of a CD44 antibody,
0.02% polysorbate 20,
240 mM Trehalose,
20 mM L-histidine,

at pH 6.0;

or
10 mg/mL of a CD44 antibody,
0.02% polysorbate 20,
190 mM sucrose,
20 mM L-arginine,

at pH 5.5.

In another preferred aspect, the invention relates to a CD44 antibody formulation, wherein it is:
10 mg/mL of a CD44 antibody,
150 mM NaCl,
0.01% of polysorbate 80,
20 mM of a L-histidine,
at a pH of 6.

In another aspect, the invention relates to the CD44 antibody formulation as described herein, for the use as medicament in treating diseases modulated by the CD44 receptor. In another aspect, the invention relates to the CD44 formulation as described herein for use as medicament in treating a disease selected from the group consisting of breast cancer, preferably triple negative breast cancer, hepatocellular carcinoma, acute myeloid leukemia and squameous cell carcinoma, preferably head and neck squamous cell carcinoma (HNSCC).

### DETAILED DESCRIPTION OF EMBODIMENTS OF THE INVENTION

### I. Definitions

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The terms “anti-CD44 antibody” and “an antibody that binds to CD44” refer to an antibody that is capable of binding CD44 with sufficient affinity such that the antibody is useful as a diagnostic and/or therapeutic agent in targeting CD44. In one embodiment, the extent of binding of an anti-CD44 antibody to an unrelated, non-CD44 protein is less than about 10% of the binding of the antibody to CD44 as measured, e.g., by a radioimmunoassay (RIA). In certain embodiments, an antibody that binds to CD44 has a dissociation constant (Kd) of ≦1 μM, ≦100 nM, ≦10 nM, ≦1 nM, ≦0.1 nM, ≦0.01 nM, or ≦0.001 nM (e.g. 10⁻⁸ M or less, e.g. from 10⁻⁸M to 10⁻¹³M, e.g., from 10⁻⁹M to 10⁻¹³ M). In certain embodiments, an anti-CD44 antibody binds to an epitope of CD44 that is conserved among CD44 from different species.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below. “Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

An “effective amount” of an agent, e.g., a pharmaceutical formulation, refers to an amount effective, at dosages and for periods of time necessary, to achieve the desired therapeutic or prophylactic result.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, Md. (1991).) With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008).) Unless otherwise indicated, HVR residues and other residues in the variable domain (e.g., FR residues) are numbered herein according to Kabat et al., supra.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-CD44 antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell. The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “cancer” as used herein refers to cancers or tumors which express the tumor antigen to which the afocusylated antibody is specifically binding. Such cancers includes lymphomas, lymphocytic leukemias, preferably acute or chronic lymphocytic leukemia, myeloid leukemia, preferably acute or chronic myeloid leukemia, lung cancer, non small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, Ewing's sarcoma, pituitary adenoma, including refractory versions of any of the above cancers, or a combination of one or more of the above cancers. Preferably, said cancer is breast cancer, preferably triple negative breast cancer, hepatocellular carcinoma, acute myeloid leukemia or squameous cell carcinoma, preferably head and neck squamous cell carcinoma (HNSCC). The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject., A pharmaceutically acceptable carrier includes, but is not limited to, a buffer, excipient, stabilizer, or preservative.

The term “CD44,” as used herein, refers to any native CD44 from any vertebrate source, including mammals such as primates (e.g. humans) and rodents (e.g., mice and rats), unless otherwise indicated. The term encompasses “full-length,” unprocessed CD44 as well as any form of CD44 that results from processing in the cell. The term also encompasses naturally occurring variants of CD44, e.g., splice variants or allelic variants. As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### II. Compositions and Methods

In one aspect, the invention is based, in part, on formulations of CD44 antibodies. In certain embodiments, antibodies that bind to CD44 are provided. Antibodies of the invention are useful, e.g., for the diagnosis or treatment of CD44-related diseases, in particular oncological diseases.

A. Exemplary Anti-CD44 Antibodies

In one aspect, the invention provides formulations of isolated antibodies that bind to CD44. In certain embodiments, an anti-CD44 antibody
mediate anti-cancer effects through ligation of epitopes present on CD44
can be used to immunoprecipitate the cognate antigen from expressing cells such as MDA-MB-231 cells

In certain aspects the invention encompasses the formulation of anti-CD44 antibodies that bind to the amino terminal domain of the extracellular domain of CD44 and/or that immunospecifically bind to one or more, preferably multiple human variants CD44 as expressed in humans. In particular aspects, the invention encompasses the formulation of the anti-CD44 antibody produced by the hybridoma deposited with the ATCC having accession number PTA-4621, and variants and/or derivatives thereof, e.g., chimeric and humanized versions of PTA-4621. The invention further encompasses the formulation of antibodies and antigen binding fragments thereof that compete for binding with PTA-4621.

The antibodies and fragments formulated according to the methods of the invention are specific for CD44, in particular CD44 as expressed on the surface of a cell, and preferably, specific for the amino terminal extracellular domain of one or more variants of human CD44, e.g., as expressed on the surface of a HNSCC cancer cell. In certain aspects the antibody formulations according to the methods disclosed herein are humanized or chimeric versions of the antibody produced by the hybridoma deposited with the ATCC having accession number PTA-4621. In specific embodiments the antibody formulations according to the methods disclosed herein, or antigen binding fragments thereof, comprise one or more of a heavy chain variable domain (VH) CDR1 having the amino acid sequence of RYWMS (SEQ ID NO:3),

a VH CDR2 having the amino acid sequence of EVNPDSTSINYTPSLKD (SEQ ID NO:4),

a VH CDR3 having the amino acid sequence of PNYYGSRYHYYAMDY (SEQ ID NO:5),

a light chain variable domain (VL) CDR1 having the amino acid sequence of RASQDINNYLN (SEQ ID NO:6),

a VL CDR2 having the amino acid sequence of YTSRLHS (SEQ ID NO:7); and

a VL CDR3 having the amino acid sequence of QQGSTLPFT (SEQ ID NO:8).

The formulation of antibodies as encompassed by the invention may also comprise two or more of, three or more of, four or more of, five or more of, and, in certain aspects, all of a VH CDR1 having the amino acid sequence of SEQ ID NO:3, a VH CDR2 having the amino acid sequence of SEQ ID NO:4, a VH CDR3 having the amino acid sequence of SEQ ID NO:5, a VL CDR1 having the amino acid sequence of SEQ ID NO:6, a VL CDR2 having the amino acid sequence of SEQ ID NO:7 and a VL CDR3 having the amino acid sequence of SEQ ID NO:8. The antibodies and antigen binding fragment formulations according to the methods of the invention may comprise

a VH domain having the amino acid sequence of

(SEQ ID NO: 1)
EVKLLESGGGLVQPGGSLKLSCATSGFDFSRYWMSWVRQAPGKGLEWIGE
VNPDSTSINYTPSLKDQFIISRDNAKNTLDLQMSKVSSEDTALYYCTRPN
YYGSRYHYYAMDYWGQGTSVTVSS

and/or a VL domain having the amino acid sequence of

(SEQ ID NO: 2)
DIQMTQTTSSLSVSLGDRVTINCRASQDINNYLNWYQQKPDGTVKLLIYY
TSRLHSGVPSRFSGSGSGTDFSLTISNLEKEDVATYFCQQGSTLPFTFGS
GTKLEIK.

In certain aspects the invention encompasses the formulation of an anti-CD44 antibody or antigen binding fragment comprising a VH domain having the amino acid sequence of SEQ ID NO:1 and a VL domain having the amino acid sequence of SEQ ID NO:2.

The invention further encompasses the formulation of an anti-CD44 antibody, or antigen binding fragments thereof, wherein the anti-CD44 antibody is humanized. In some aspects, the humanized anti-CD44 antibody or antigen binding fragment comprises

a VH domain having the amino acid sequence of

(SEQ ID NO: 9)
EVQLVESGGGLVQPGGSLRLSCAASGFDFSRYWMSWVRQAPGKGLVWVGE
VNPDSTSINYTPSLKDRFTISRDNAKNTLYLQMNSLRAEDTAVYYCTRPN
YYGSRYHYYAMDYWGQGTLVTVSS
or
(SEQ ID NO: 10)
EVQLVESGGGLVQPGGSLRLSCATSGFDFSRYWMSWVRQAPGKGLVWIGE
VNPDSTSINYTPSLKDQFTISRDNAKNTLYLQMNSLRAEDTAVYYCTRPN
YYGSRYHYYAMDYWGQGTLVTVSS

and/or a VL domain having the sequence of

(SEQ ID NO: 11)
DIQMTQSPSSLSASVGDRVTITCRASQDINNYLNWYQQKPGKAPKLLIYY
TSRLHSGVPSRFSGSGSGTDFTFTISSLQPEDIATYYCQQGSTLPFTFGQ
GTKLEIK.

In a particular aspect, the invention provides for a formulation of a humanized anti-CD44 antibody or antigen binding fragment thereof, wherein said humanized anti-CD44 antibody comprises a VH domain having the amino acid sequence of SEQ ID NO:9 and a VL domain having the amino acid sequence of SEQ ID NO:11, or wherein said anti-CD44 antibody comprises a VH domain having the amino acid sequence of SEQ ID NO:10 and a VL domain having the amino acid sequence of SEQ ID NO:11. In particular aspects the subject is a human. The invention also provides for the formulation of a humanized anti-CD44 antibody or antigen binding fragment thereof, wherein said humanized anti-CD44 antibody comprises a VH domain having the amino acid sequence of SEQ ID NO:9 and a VL domain having the amino acid sequence of SEQ ID NO:11, or wherein said anti-CD44 antibody comprises a VH domain having the amino acid sequence of SEQ ID NO:10 and a VL domain having the amino acid sequence of SEQ ID NO:11.

In certain aspects, the antibody or antibody fragment formulation in accordance with the methods of the invention recognizes the epitope within the amino terminal domain of the extracellular region of CD44 having the amino acid sequence of AFDGPITITIV (SEQ ID NO:12).

The anti-CD44 antibody formulations according to the invention may be conjugated to active moieties such as therapeutic or reporter moieties. The anti-CD44 antibodies may also be conjugated to hematogeneous cells from the subject. In certain aspects the therapeutic moieties are moieties known in the art to be beneficial for the treatment, prevention or diagnosis of CD44-related diseases, e.g. cancer, such as, but not limited to, a cytotoxin, an enzyme, a radioactive element, a cytokine, an antimetabolite or an interferon.

The invention also encompasses the use of anti-CD44 antibody or antigen binding fragment formulations thereof as single agent therapies or in combination with one or more other chemotherapeutic or diagnostic agents and/or therapies. Accordingly, the invention encompasses the use of anti-CD44 antibody formulations (e.g., formulations of humanized or chimeric antibodies), or antigen binding fragments thereof, in combination with a standard or experimental treatment regimen for CD44-related disease, e.g. cancer, in particular e.g. Head and Neck Sqameous Cell Carcinoma (HNSCC (e.g., chemotherapy, radioimmunotherapy, or radiotherapy)). Examples of therapeutic agents that are particularly useful in combination with formulations of a CD44-specific antibody or an antigen-binding fragment thereof, according to the methods disclosed herein for the prevention, treatment, or diagnosis of CD44-related disease, e.g. cancer, in particular e.g. HNSCC, include, but are not limited to chemotherapeutics as known in the art, alkylating agents, antimetabolites, natural products, and hormones. The combination therapies as disclosed herein may enable lower dosages of a formulation of an anti-CD44 antibody or an antigen-binding fragment thereof and/or less frequent administration of a formulation of the anti-CD44 antibody or an antigen-binding fragment thereof to a subject with a CD44-related disease, e.g. cancer, in particular e.g. HNSCC, to achieve a therapeutic or prophylactic effect. According to the methods disclosed herein, the formulations of anti-CD44 antibodies or antigen-binding fragments thereof may be co-administered, concurrently administered, and/or sequentially administered with the one or more other chemotherapeutic or diagnostic agents, or may be administered in conjunction with radiation therapy or surgery. Administration of the formulations of the antibodies, fragments and/or additional agents or therapies, may be by any means known in the art to be suitable for delivery of the particular agent or therapy to the tumor site. Such administration may be parenteral (e.g., IV, intramuscular or subcutaneous administration), oral or, in certain aspects, direct delivery to the tumor site or to the site of suspected tumor occurrence or recurrence. The formulation of the antibodies, fragments and/or compositions may also be administered as a sustained release formulation.

In certain aspects, the anti-CD44 antibodies and antigen binding fragments thereof comprised in the formulations as disclosed herein may comprise an amino acid sequence of a VH domain and/or VL domain that is at least 45%, at least 50%, at least 55%, at least 60%, at least 65%, at least 70%, at least 75%, at least 80%, at least 85%, at least 90%, at least 95%, or at least 99% identical to the amino acid sequence of the VH domain and/or VL domain of the mouse monoclonal antibody produced by the hybridoma deposited with the ATCC having accession number PTA-4621, provided that the anti-CD44 antibodies or fragments exhibit binding specificity to CD44 and/or compete for binding with PTA-4621. The invention also encompasses the formulation of anti-CD44 antibodies or antigen binding fragments thereof that comprise an amino acid sequence of a VH domain and/or VL domain that is at least 45%, at least 50%, at least 55%, at least 60%, at least 65%, at least 70%, at least 75%, at least 80%, at least 85%, at least 90%, at least 95%, or at least 99% identical to the VH domain having the amino acid sequence of SEQ ID NO:1 and/or the VL domain having the amino acid sequence of SEQ ID NO:2, provided that the anti-CD44 antibodies or fragments exhibit binding specificity to CD44 and/or compete for binding with PTA-4621. In certain aspects, the present invention encompasses formulations of antibodies or fragments thereof that specifically bind CD44 and that comprise an amino acid sequence of one or more CDRs that is at least 45%, at least 50%, at least 55%, at least 60%, at least 65%, at least 70%, at least 75%, at least 80%, at least 85%, at least 90%, at least 95%, or at least 99% identical to the amino acid sequence of one or more CDRs of the mouse monoclonal antibody produced by the hybridoma deposited with the ATCC having accession number PTA-4621, provided that said antibodies or fragments exhibit binding specificity to CD44 and/or compete for binding with PTA-4621. The invention also encompasses the formulation of anti-CD44 antibodies or antigen binding fragments thereof that comprise an amino acid sequence of one or more CDRs that are at least 45%, at least 50%, at least 55%, at least 60%, at least 65%, at least 70%, at least 75%, at least 80%, at least 85%, at least 90%, at least 95%, or at least 99% identical to one or more of a VH CDR1 having the amino acid sequence of SEQ ID NO:3, a VH CDR2 having the amino acid sequence of SEQ ID NO:4, a VH CDR3 having the amino acid sequence of SEQ ID NO:5, a VL CDR1 having the amino acid sequence of SEQ ID NO:6, a VL CDR2 having the amino acid sequence of SEQ ID NO:7 and a VL CDR3 having the amino acid sequence of SEQ ID NO:8, provided that provided that said antibodies or fragments exhibit binding specificity to CD44 and/or competes for binding with PTA-4621.

The present invention also encompasses the formulation of humanized antibodies and antibody fragments specific for CD44 or that compete for binding with antibody PTA-4621, in which one or more regions of one or more CDRs of the heavy and/or light chain variable regions of a human antibody (the recipient antibody) have been substituted by analogous parts of one or more CDRs of a donor monoclonal antibody which specifically binds CD44, e.g., of the murine monoclonal antibody produced by clone PTA-4621. Preferably, the humanized antibody specifically binds to the same epitope as the donor murine antibody. It will be appreciated by one skilled in the art that the invention encompasses CDR grafting of antibodies in general. In particular aspects, the present invention encompasses the formulation of humanized antibodies or antibody fragments that specifically bind CD44 and/or compete for binding with PTA-4621, which humanized antibodies or antibody fragments comprise an amino acid sequence of a VH domain and/or VL domain that is at least 45%, at least 50%, at least 55%, at least 60%, at least 65%, at least 70%, at least 75%, at least 80%, at least 85%, at least 90%, at least 95%, or at least 99% identical to the VH domain having the amino acid sequence of SEQ ID NO:9 or SEQ ID NO:10 and/or the VL domain having the amino acid sequence of SEQ ID NO:11.

The present invention also encompasses the formulation of a CD44 antibody as disclosed in WO2008/144890, preferably an antibody selected from the group of the murine antibody H460-16-2 (hereinafter “<muMabCD44>”), the chimeric antibody (ch)ARH460-16-2-IgG2 (hereinafter “<ch1MabCD44>”), the chimeric antibody (ch)ARH460-16-2-IgG1 (hereinafter “<ch2MabCD44>”), the chimeric antibody (ch)ARH460-16-2 (VK0VH0) (hereinafter “<ch3MabCD44>”), and/or the humanized antibody (hu)ARH460-16-2 variant HV1/KV1 (hereinafter “<hu1MabCD44>”), and/or the humanized antibody (hu)ARH460-16-2 variant HV2/KV1 (hereinafter “<hu2MabCD44>”), as disclosed also in WO2008/144890, incorporated by reference in its entirety, preferably for the antibodies as mentioned before and disclosed e.g. on page 29, lines 17-18 as well as in the examples of said patent application.

The anti-CD44 antibodies and antigen binding fragments thereof as in the formulations disclosed herein may be encoded by a nucleotide sequence that hybridizes under stringent conditions to the nucleotide sequence encoding a VH domain, the complete heavy chain, a VL domain or the complete light chain of the murine monoclonal antibody PTA-4621. In other aspects, the invention encompasses the formulation of a an anti-CD44 antibody or antigen binding fragment thereof comprising a VH domain that is encoded by a nucleic acid sequence that hybridizes under stringent conditions to a nucleic acid sequence encoding the amino acid sequence of SEQ ID NO:1, SEQ ID NO:9 or SEQ ID NO:10. In other aspects, the invention encompasses the formulation of a an anti-CD44 antibody or antigen binding fragment thereof comprising a VL domain that is encoded by a nucleic acid sequence that hybridizes under stringent conditions to a nucleic acid sequence encoding the amino acid sequence of SEQ ID NO:2 or SEQ ID NO:11. The invention encompasses the formulation of an anti-CD44 antibody or antigen binding fragment thereof comprising one or more CDRs encoded by a nucleotide sequence that hybridizes under stringent conditions to the nucleotide sequence encoding one or more CDRs of the murine monoclonal antibody PTA-4621, or the nucleic acid sequence encoding one or more of VH CDR1 having the amino acid sequence of SEQ ID NO:3, a VH CDR2 having the amino acid sequence of SEQ ID NO:4, a VH CDR3 having the amino acid sequence of SEQ ID NO:5, a VL CDR1 having the amino acid sequence of SEQ ID NO:6, a VL CDR2 having the amino acid sequence of SEQ ID NO:7 and a VL CDR3 having the amino acid sequence of SEQ ID NO:8. Stringent hybridization conditions include, but are not limited to, (i) hybridization to filter-bound DNA in 6× sodium chloride/sodium citrate (SSC) at about 45oC, followed by one or more washes in 0.2×SSC/0.1% SDS at about 50-65oC; (ii) highly stringent conditions such as hybridization to filter-bound DNA in 6×SSC at about 45oC, followed by one or more washes in 0.1×SSC/0.2% SDS at about 60oC, or (iii) any other stringent hybridization conditions known to those skilled in the art.

The invention also relates to the use of a vector comprising the nucleic acids disclosed herein (i.e., nucleic acids encoding VH domains, VL domains and/or one or more CDR of anti-CD44 antibody or antigen binding fragments). In certain embodiments, said vector is an expression vector and the nucleic acid sequences encoding the amino acid sequences as described herein are operably joined with nucleic acid regulatory sequences or sequences encoding amino acid regulatory sequences (e.g., transcription, translation, translocation signals) necessary for the proper expression of the encoded amino acid sequences. The invention further provides host cells containing the vectors or nucleotide sequences encoding the antibodies or antibody fragments as in the formulations disclosed herein.

Other objects and advantages of this invention will become apparent from the following description wherein are set forth, by way of illustration and example, certain embodiments of this invention.

In a further aspect of the invention, an anti-CD44 antibody according to any of the above embodiments is a monoclonal antibody, including a chimeric, humanized or human antibody. In one embodiment, an anti-CD44 antibody is an antibody fragment, e.g., a Fv, Fab, Fab′, scFv, diabody, or F(ab′)₂ fragment. In another embodiment, the antibody is a full length antibody, e.g., an intact antibody or other antibody class or isotype as defined herein.

In a further aspect, an anti-CD44 antibody according to any of the above embodiments may incorporate any of the features, singly or in combination, as described in Sections 1-7 below:

1. Antibody Affinity

In certain embodiments, an antibody provided herein has a dissociation constant (Kd) of ≦1 μM, ≦100 nM, ≦10 nM, ≦1 nM, ≦0.1 nM, ≦0.01 nM, or ≦0.001 nM (e.g. 10⁻⁸ M or less, e.g. from 10⁻⁸M to 10⁻¹³M, e.g., from 10⁻⁹M to 10⁻¹³ M).

In one embodiment, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays. According to another embodiment, Kd is measured using surface plasmon resonance assays using a BIACORE®-2000 or a BIACORE®-3000 (BIAcore, Inc., Piscataway, N.J.) at 25° C. with immobilized antigen CM5 chips at ˜10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

1. Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthiin, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

2. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof. In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity. Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409;

Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

3. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

4. Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

6. Multispecific Antibodies

In certain embodiments, an antibody provided herein is a multispecific antibody, e.g. a bispecific antibody. Multispecific antibodies are monoclonal antibodies that have binding specificities for at least two different sites. In certain embodiments, one of the binding specificities is for CD44 and the other is for any other antigen. In certain embodiments, bispecific antibodies may bind to two different epitopes of CD44 Bispecific antibodies may also be used to localize cytotoxic agents to cells which express CD44. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to CD44 as well as another, different antigen (see, US 2008/0069820, for example).

7. Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

a) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 1 under the heading of “conservative substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; Met; Ala; | Ile |
| | Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |
| | Norleucine | |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

b) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

c) Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc(RIII only, whereas monocytes express Fc(RI, Fc(RII and Fc(RIII FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)). Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000). Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. No. 5,648,260; U.S. Pat. No. 5,624,821; and WO 94/29351 concerning other examples of Fc region variants.

d) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

e) Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide copolymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

B. Recombinant Methods and Compositions

Antibodies may be produced using recombinant methods and compositions, e.g., as described in U.S. Pat. No. 4,816,567. In one embodiment, isolated nucleic acid encoding an anti-CD44 antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti CD44 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-CD44 antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

### III. Antibody Formulations

Therapeutic formulations of the antibodies used in accordance with the present invention are prepared for storage by mixing an antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed.

The term “surfactant” as used herein denotes a pharmaceutically acceptable surface-active agent. In the formulation of the invention, the amount of surfactant is described as a percentage expressed in weight/volume. The most commonly used weight/volume unit is mg/mL. Suitable pharmaceutically acceptable surfactants comprise but are not limited to polyethylen-sorbitan-fatty acid esters, polyethylene-polypropylene glycols, polyoxyethylene-stearates and sodium dodecyl sulphates. Preferred polyethylen-sorbitan-are polyethylen(20)-sorbitan-esters (synonym to polysorbate 20, sold under the trademark Tween 20™) and polyoxyethylene(20)sorbitanmonooleate (synonym to polysorbate 80 sold under the trademark Tween 80™). Preferred polyethylene-polypropylene glycols are those sold under the names Pluronic® F68 or Poloxamer 188™. Preferred polyoxyethylene-stearates are those sold under the trademark Myrj™. Preferred polyoxyethylene monolauryl ether are those sold under the trademark Brij™. When polyethylen-sorbitan-polyethylen(20)-sorbitan-esters (Tween 20™) and polyoxyethylene(20)sorbitanmonooleate (Tween 80™) are used they are preferably used in an amount of about 0.01% to about 0.06%, more preferably of about 0.02% to about 0.04% and most preferably about 0.03% w/v.

When amounts are expressed as “mM” herein, it means the amount of a given agent that will yield the recited concentration of agent in mM.

The term “buffer” as used herein denotes a pharmaceutically acceptable buffer. Suitable pharmaceutically acceptable buffer comprise but are not limited to histidine-buffers, citrate-buffers, succinate-buffers, acetate-buffers and phosphate-buffers. Preferred buffers comprise L-histidine or mixtures of L-histidine with L-histidine hydrochloride with isotonicity agents and potentially pH adjustment with an acid or a base known in the art. The above-mentioned histidine-buffers are generally used in an amount of about 1 mM to about 100 mM, preferably of about 5 mM to about 50 mM and still more preferably of about 20 mM. Independently from the buffer used, the pH will be adjusted at a value comprising about 4.5 to about 7.0 and preferably about 5.0 to about 6.0 and most preferably about 5.5 by adjustment with an acid or base known in the art or by using adequate mixtures of buffer components or both.

The term “isotonicity agents” as used herein denotes pharmaceutically acceptable isotonicity agents. Isotonicity agents are used to provide an isotonic formulation. An isotonic formulation is liquid or liquid reconstituted from a solid form, e.g. a lyophilized form and denotes a solution having the same tonicity as some other solution with which it is compared, such as physiologic salt solution and the blood serum. Suitable isotonicity agents comprise but are not limited to salts, including but not limited to sodium chloride (NaCl) or potassium chloride, sugars including but not limited to glucose, sucrose, trehalose or and any component from the group of amino acids, sugars, salts and combinations thereof. Isotonicity agents are generally used in a total amount of about 5 mM to about 350 mM.

The term “liquid” as used herein in connection with the formulation according to the invention denotes a formulation which is liquid at a temperature of at least about 2 to about 8° C. Unless otherwise noted, the term “formulation” herein refers to a liquid formulation.

The term “lyophilized” as used herein in connection with the formulation according to the invention denotes a formulation which is dried by freezing the formulation and subsequently subliming the ice from the frozen content by any freeze-drying methods known in the art, for example commercially available freeze-drying devices. When amounts of lyophilized formulation components are set forth, they refer to the concentration of the component after the lyophilized formulation has been reconstituted in water.

The term “salts” as used herein denotes a salt in an amount of about 1 mM to about 500 mM. Non-limiting examples of salts include salts of any combinations of the cations sodium potassium, calcium or magnesium with anions chloride, phosphate, citrate, succinate, sulphate or mixtures thereof.

The term “amino acid” as used herein denotes an amino acid in an amount of about 1 to about 200 mg/mL comprising but not limited to arginine, glycine, ornithine, lysine, histidine, glutamic acid, asparagic acid, isoleucine, leucine, alanine, phenylalanine, tyrosine, tryptophane, methionine, serine, proline. Preferred is methionine. More preferred is methionine at about 5 mM to about 15 mM. Most preferred is methionine at about 10 mM. Also preferred is arginine HCl, more preferred is arginine HCl at about 100 mM to about 200 mM. Most preferred is arginine HCl at about 150 mM.

The term “sugar” as used herein denotes a pharmaceutically acceptable sugar used in an amount of about 25 mM to about 500 mM. Suitable sugars comprise but are not limited monosaccharides and disaccharides. Non-limiting examples of sugars according to the invention include trehalose, sucrose, mannitol, sorbitol, lactose, glucose, mannose, maltose, galactose, fructose, sorbose, raffinose, glucosamine, N-methylglucosamine (also referred to as “meglumine”), galactosamine and neuraminic acid and combinations thereof. Preferred is trehalose. More preferred is trehalose at about 200 mM to about 300 mM. Most preferred is trehalose at about 240 mM.

The term “stabilizer” refers to pharmaceutically acceptable stabilizers, like for example but not limited to amino acids and sugars as described in the above sections as well as commercially available cyclodextrins and dextrans of any kind and molecular weight as known in the art. Preferred are amino acids and/or sugars.

The term “antioxidant” denotes a pharmaceutically acceptable antioxidant. This may include excipients such as methionine, benzylalcohol or any other excipient used to minimize oxidation.

As mentioned above, in one aspect, the invention relates to a CD44 antibody formulation comprising:
about 5 to about 30 mg/mL of a CD44 antibody as described herein,
   about 5 to about 50 mM of a buffer,
   at a pH in the range from about 4.5 to about 7.0.

A preferred formulation according to the invention comprises:
about 5 to about 30 mg/mL of a CD44 antibody as described herein,
about 10 to about 30 mM of a buffer,
at a pH in the range from about 5.0 to about 6.5.

A preferred formulation according to the invention further comprises about 0.005% to about 0.06% of at least one surfactant.

A preferred formulation according to the invention further comprises at least one stabilizer.

Another preferred formulation according to the invention comprises:
about 5 to about 30 mg/mL of a CD44 antibody as described herein,
at least one further stabilizer, and
about 0.02 to about 0.04% of at least one surfactant, and
about 15 to about 25 mM of a buffer,
at a pH from about 5.0 to about 6.0.

In a preferred formulation according to the invention, the stabilizers are selected from the group of sugars, amino acids and salts.

The formulation of the invention can comprise a sugar in an amount of about 25 mM to about 500 mM. Suitable sugars can be selected from the group consisting of trehalose, saccharose, lactose, glucose, mannose, maltose, galactose, fructose, sorbose, raffinose, glucosamine, N-Methylglucosamine, galactosamine, neuraminic acid and combinations thereof. In a more preferred formulation according to the invention, the stabilizer is trehalose. In an even more preferred formulation according to the invention, the formulation comprises about 200 mM to about 300 mM trehalose. In another more preferred formulation according to the invention, the stabilizer is methionine. In an even more preferred formulation according to the invention, the formulation comprises about 5 mM to about 30 mM methionine. In another more preferred formulation according to the invention, the formulation comprises the stabilizers trehalose and methionine. In an even more preferred formulation according to the invention, the formulation comprises about 230 mM to about 250 mM trehalose and about 5 mM to about 15 mM methionine. In another even more preferred formulation according to the invention, the formulation comprises about 240 mM trehalose. In another even more preferred formulation according to the invention, the formulation comprises about 10 mM methionine.

A preferred formulation according to the invention comprises:
about 5 to about 30 mg/mL of a of a CD44 antibody as described

herein,
about 10 to about 30 mM of a buffer,
about 200 mM to about 300 mM trehalose
at a pH in the range from about 5.0 to about 6.5.

A preferred formulation according to the invention comprises:
about 5 to about 30 mg/mL of a of a CD44 antibody as described

herein,
about 10 to about 30 mM of a buffer,
about 150 mM to about 300 mM sucrose
at a pH in the range from about 5.0 to about 6.5.

Another preferred formulation according to the invention comprises:
about 5 to about 30 mg/mL of a of a CD44 antibody as described

herein,
about 10 to about 30 mM of a buffer,
about 100 mM to about 200 mM NaCl
at a pH in the range from about 5.0 to about 6.5.

Another preferred formulation according to the invention comprises:
about 5 to about 30 mg/mL of a of a CD44 antibody as described

herein,
about 10 to about 30 mM of a buffer,
about 200 mM to about 300 mM trehalose
5 mM to about 50 mM amino acid
at a pH in the range from about 5.0 to about 6.5.

A preferred formulation according to the invention, is in a liquid form, in a lyophilized form or in a liquid form reconstituted from a lyophilized form.

The preferred formulation according to the invention, can be administered by intravenous (i.v.), subcutaneous (s.c.) or any other parental administration means such as those known in the pharmaceutical art.

The formulation according to the invention also comprises the following specific formulations.

A most preferred liquid formulation according to the invention is:
about 10 mg/mL of a of a CD44 antibody as described herein,
about 20 mM L-histidine,
about 150 mM sodium chloride (NaCl),
about 0.01% polysorbate 80,

at a pH of about 6.0;

or
about 10 mg/mL of a of a CD44 antibody as described herein,
about 20 mM L-histidine,
about 240 mM trehalose,
about 0.02% polysorbate 20,

at pH of about 6.0;

or
about 10 mg/mL of a CD44 antibody as described herein,
about 20 mM citrate,
about 190 mM sucrose,
about 20 mM L-arginine,
about 0.02% polysorbate 20,

at pH of about 5.5;

or
about 25 mg/mL of a CD44 antibody as described herein,
about 20 mM L-histidine,
about 140 mM NaCl,
about 0.02% Poloxamer 188,

at pH of about 5.5;

or
about 25 mg/mL of a CD44 antibody,
about 20 mM L-histidine,
about 140 mM NaCl,
about 0.02% Polysorbate 20,

at a pH of about 5.5;

or
about 25 mg/mL of a CD44 antibody,
about 20 mM L-histidine,
about 140 mM NaCl,
about 0.02% Polysorbate 20,

at a pH of about 6.0;

or
about 25 mg/mL of a CD44 antibody,
about 20 mM L-histidine,
about 140 mM NaCl,
about 0.02% Poloxamer 188,

at a pH of about 6.0;

or
about 25 mg/mL of a CD44 antibody,
about 20 mM L-histidine,
about 240 mM Sucrose,
about 0.02% Poloxamer 188,

at a pH of about 5.5;

or
about 25 mg/mL of a CD44 antibody,
about 20 mM L-histidine,
about 240 mM Sucrose,
about 0.02% Poloxamer 188,

at a pH of about 6.0.

A most preferred lyophilized formulation according to the invention is:
about 10 mg/mL of a of a CD44 antibody as described herein,
about 20 mM L-histidine,
about 240 mM trehalose,
about 0.02% polysorbate 20,

at pH of about 6.0;
or
about 10 mg/mL of a of a CD44 antibody as described herein,
about 20 mM L-histidine,
about 190 mM sucrose,
about 20 mM L-arginine,
about 0.02% polysorbate 20,

at pH of about 5.5.

In another preferred aspect, any one of the preferred formulations as above comprises an exact quantity or exact quantities of one or more components as comprised therein and/or an exact pH value. In other words, one or more of the terms “about” are deleted in this other preferred aspect of the invention.

Therefore, preferred formulations are as follows.

Preferred liquid formulation according to the invention comprise:
10 mg/mL of a CD44 antibody as described herein,
20 mM L-histidine,
150 mM NaCl,
0.01% polysorbate 80,

at a pH of 6.0;

or
10 mg/mL of a CD44 antibody as described herein,
20 mM L-histidine,
240 mM trehalose,
0.02% polysorbate 20,

at a pH of 6.0;

or
10 mg/mL of a CD44 antibody as described herein,
20 mM citrate,
190 mM sucrose,
20 mM L-arginine,
0.02% polysorbate 20,

at pH 5.5;

or
25 mg/mL of a CD44 antibody as described herein,
20 mM L-histidine,
140 mM NaCl,
0.02% Poloxamer 188,

at pH of 5.5;

or
25 mg/mL of a CD44 antibody,
20 mM L-histidine,
140 mM NaCl,
0.02% Polysorbate 20,

at a pH of 5.5;

or
25 mg/mL of a CD44 antibody,
20 mM L-histidine,
140 mM NaCl,
0.02% Polysorbate 20,

at a pH of 6.0;

or
25 mg/mL of a CD44 antibody,
20 mM L-histidine,
140 mM NaCl,
0.02% Poloxamer 188,

at a pH of 6.0;

or
25 mg/mL of a CD44 antibody,
20 mM L-histidine,
240 mM Sucrose,
0.02% Poloxamer 188,

at a pH of 5.5;

or
25 mg/mL of a CD44 antibody,
20 mM L-histidine,
240 mM Sucrose,
0.02% Poloxamer 188,

at a pH of 6.0.

Preferred lyophilized formulations according to the invention comprises:
10 mg/mL of a CD44 antibody as described herein,
20 mM L-histidine,
240 mM trehalose,
0.02% polysorbate 20,

at pH 6.0;

or
10 mg/mL of a CD44 antibody as described herein,
20 mM L-histidine,
190 mM sucrose,
20 mM L-arginine,
0.02% polysorbate 20,

at a pH of 5.5.

Another preferred formulation according to the invention comprises:
10 mg/mL of a CD44 antibody as described herein,
150 mM NaCl,
20 mM of a L-histidine,
0.01% of polysorbate 80,
at a pH of 6.

Another preferred formulation according to the invention comprises:
25 mg/mL of a CD44 antibody as described herein,
20 mM L-histidine,
140 mM NaCl,
0.02% Poloxamer 188,
at a pH of 5.5.

In another preferred aspect, the CD44 antibody as in any of the preferred formulations above comprises one or more of the CDRs selected from the group of SEQ ID NO:3, SEQ ID NO:4, SEQ ID NO:5, SEQ ID NO:6, SEQ ID NO:7, or SEQ ID NO:8.

In another preferred aspect, the CD44 antibody as in any of the preferred formulations above is humanized.

In another preferred aspect, the CD44 antibody as in any of the preferred formulations above comprises SEQ ID NO:9.

In another preferred aspect, the CD44 antibody as in any of the preferred formulations above comprises SEQ ID NO:10.

In another preferred aspect, the CD44 antibody as in any of the preferred formulations above further comprises SEQ ID NO:11.

In another preferred aspect, the CD44 antibody as in any of the preferred formulations above is a CD44 antibody as disclosed in WO2008/144890, preferably an antibody selected from the group of the murine antibody H460-16-2 (hereinafter “<muMabCD44>”), the chimeric antibody (ch)ARH460-16-2-IgG2 (hereinafter “<ch1MabCD44>”), the chimeric antibody (ch)ARH460-16-2-IgG1 (hereinafter “<ch2MabCD44>”), the chimeric antibody (ch)ARH460-16-2 (VK0VH0) (hereinafter “<ch3MabCD44>”), and/or the humanized antibody (hu)ARH460-16-2 variant HV1/KV1 (hereinafter “<hu1MabCD44>”), and/or the humanized antibody (hu)ARH460-16-2 variant HV2/KV1 (hereinafter “<hu2MabCD44>”), as disclosed also in WO2008/144890, incorporated by reference in its entirety, preferably for the antibodies as mentioned before and disclosed e.g. on page 29, lines 17-18 as well as in the examples of said patent application.

The formulation of the invention can further comprise one or more of the following ingredients: antioxidants, ascorbic acid, glutathione, preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); cyclodextrin, e.g. hydroxypropyl-β-cyclodextrin, sulfobutylethyl-β-cyclodextrin, β-cyclodextrin, polyethyleneglycol, e.g. PEG 3000, 3350, 4000, or 6000; low molecular weight (less than about 10 residues) polypeptides; proteins, such as serum albumin, gelatin, or immunoglobulins; chelating agents such as EDTA; salt-forming counter-ions such as sodium; and metal complexes (e.g. Zn-protein complexes).

The formulation of the invention can further comprise one or more stabilizers as defined hereinabove and ingredients also known in the art as “lyoprotectants” such as sugars, sugar alcohols, amino acids and dextrans as known in the art.

In a most preferred embodiment of the formulation according to this invention, the formulation is a liquid form and comprises:
about 10 mg/mL of a of a CD44 antibody as described herein,
about 0.01% polysorbate 80,
about 150 mM NaCl,
about 20 mM L-histidine,
at pH of about 6.0.

In another most preferred embodiment of the formulation according to this invention, the formulation is a liquid form and comprises:
about 25 mg/mL of a CD44 antibody,
about 20 mM L-histidine,
about 140 mM NaCl,
about 0.02% Poloxamer 188,

at a pH of about 5.5.

These formulations showed good stability upon storage for approximately 8 weeks at 2-8° C. and 25° C. without formation of visible particles. Similar results were observed in longer stability tests. Shaking and multiple freezing-thawing steps were applied to the liquid formulation to simulate physical stress conditions that potentially occur during manufacturing or transportation of the drug product.

The invention of a liquid formulation according to this embodiment is advantageous as it will facilitate ease of use for the health care provider as no reconstitution step is required and with the higher protein concentration fewer vials per patient will be required.

Liquid and lyophilized drug product formulations for intravenous administration according to the invention were developed as follows.

### EXAMPLES

### Preparation of Liquid Formulations

For the preparation of the liquid formulations a humanized anti-CD44 monoclonal antibody huMAb-CD44 having VH and VL as described in WO2008/144890 was buffer-exchanged against a diafiltration buffer containing the anticipated buffer composition and where required, concentrated by diafiltration to an antibody concentration of approximately 40 mg/mL. After completion of the diafiltration operation, the excipients (e.g. trehalose) were added as stock solutions to the antibody solution. The surfactant was then added as a 50 to 200-fold stock solution. Finally the protein concentration was adjusted with a buffer to the final huMAb-CD44 concentration of approximately 10 mg/mL.

All formulations were sterile-filtered through 0.22 μm low protein binding filters and aseptically filled into sterile 6 mL glass vials closed with ETFE (Copolymer of ethylene and tetrafluoroethylene)-coated rubber stoppers and alucrimp caps. The fill volume was approximately 2.4 mL. These formulations were stored at different climate conditions (5° C., 25° C. and 40° C.) for different intervals of time and stressed by shaking (1 week at a shaking frequency of 200 min⁻¹ at 5° C. and 25° C.) and freeze-thaw stress methods. The samples were analyzed before and after applying the stress tests by the analytical methods 1) UV spectrophotometry, 2) Size Exclusion Chromatography (SEC), 3) by Ion exchange chromatography (IEC), 4) by turbidity of the solution and 5) for visible particles.

UV spectroscopy, used for determination of protein content, was performed on a Perkin Elmer λ 35 UV spectrophotometer in a wavelength range from 240 nm to 400 nm. Neat protein samples were diluted to approximately 0.5 mg/mL with the corresponding formulation buffer. The protein concentration was calculated according to equation 1.

$$Protein content = \frac{A ( 280 ) - A ( 320 ) \times dil . factor}{ɛ 〈 {cm}^{2} \text{/} mg 〉 \times d 〈 cm 〉} \qquad Equation 1$$

The UV light absorption at 280 nm was corrected for light scattering at 320 nm and multiplied with the dilution factor, which was determined from the weighed masses and densities of the neat sample and the dilution buffer. The numerator was divided by the product of the cuvette's path length d and the extinction coefficient E.

Size Exclusion Chromatography (SEC) was used to detect soluble high molecular weight species (aggregates) and low molecular weight hydrolysis products (LMW) in the formulations. The method was performed on a Waters Alliance 2695 HPLC instrument with a Waters W2487 Dual Absorbance Detector and equipped with a TosoHaas TSK-Gel G3000SWXL column. Intact monomer, aggregates and hydrolysis products were separated by an isocratic elution profile, using 0.2M K₂HPO₄/0.25M KCL, pH 7.0 as mobile phase, and were detected at a wavelength of 280 nm.

Ion Exchange Chromatography (IEC) was performed to detect chemical degradation products altering the net charge of huMAb-CD44 in the formulations. The method used a suitable HPLC instrument equipped with a UV detector (detection wavelength 280 nm) and a Dionex ProPac WCX-10, 4 mm×250 mm. 10 mM Sodium Phosphate pH 6.5 and 10 mM Sodium Phosphate including 1M Sodium Chloride, pH 6.5, were used as mobile phases A and B, respectively, with a flow rate of 1.0 mL/min.

For the determination of the turbidity, opalescence was measured in FTU (turbidity units) using a HACH 2100AN turbidimeter at room temperature.

Samples were analyzed for visible particles by using a Seidenader V90-T visual inspection instrument.
Formulation A is a liquid formulation with the composition 10 mg/mL huMAb-CD44, 20 mM L-histidine, 150 mM NaCl, 0.01% Polysorbate 80, at pH 6.0

| | | Protein | Size Exclusion-HPLC | | | Ion Exchange-HPLC | | | | |
|---|---|---|---|---|---|---|---|---|---|---|
| Storage | | concentration | HMW | Monomer | LMW | Main Peak | Acidic Peak | Basic Peak | Turbidity | Visible |
| condition | Storage time | (mg/mL) | (%) | (%) | (%) | (%) | (%) | (%) | (FTU) | particles |
| — | Initial | 10.7 | 6.67 | 91.08 | 2.25 | 48.5 | 37.9 | 13.5 | 3.29 | Essentially |
| | | | | | | | | | | free from |
| | | | | | | | | | | particles |
| Shaking | 1 week | 10.7 | 6.76 | 91.05 | 2.19 | 48.8 | 37.9 | 13.3 | 4.80 | Free from |
| 5° C. | | | | | | | | | | particles |
| Shaking | 1 week | 10.7 | 6.56 | 91.10 | 2.35 | 47.3 | 38.4 | 14.4 | 4.78 | Free from |
| 25° C. | | | | | | | | | | particles |
| Freezing/ | — | 10.9 | 6.87 | 90.95 | 2.18 | 48.7 | 38.1 | 13.3 | 4.99 | Free from |
| Thawing | | | | | | | | | | particles |
| (5 cycles) | | | | | | | | | | |
| 5° C. | 5 weeks | 10.7 | 6.75 | 90.96 | 2.30 | 47.5 | 39.4 | 13.1 | 4.90 | Free from |
| | | | | | | | | | | particles |
| | 8 weeks | 10.8 | 6.83 | 90.70 | 2.47 | 47.9 | 38.7 | 13.4 | 4.98 | Free from |
| | | | | | | | | | | particles |
| 25° C. | 5 weeks | nd | 6.69 | 90.79 | 2.54 | 45.1 | 39.3 | 15.6 | 4.93 | Free from |
| | | | | | | | | | | particles |
| | 8 weeks | 10.8 | 6.97 | 90.09 | 2.95 | 43.1 | 40.5 | 16.4 | 5.22 | Free from |
| | | | | | | | | | | particles |
| 40° C. | 5 weeks | nd | 6.70 | 88.38 | 4.93 | 29.2 | 47.9 | 22.9 | 5.37 | Free from |
| | | | | | | | | | | particles |
| | 8 weeks | 10.8 | 6.21 | 86.99 | 6.80 | 22.07 | 52.65 | 25.27 | 5.91 | Free from |
| | | | | | | | | | | particles |

Formulation B is a liquid formulation with the composition 10 mg/mL huMAb-CD44, 20 mM L-histidine, 240 mM Trehalose, 0.02% Polysorbate 20, at pH 6.0

| | | Protein | Size Exclusion-HPLC | | | Ion Exchange-HPLC | | | | |
|---|---|---|---|---|---|---|---|---|---|---|
| Storage | Storage | concentration | HMW | Monomer | LMW | Main | Acidic | Basic | Turbidity | Visible |
| condition | time | (mg/mL) | (%) | (%) | (%) | Peak (%) | Peak (%) | Peak (%) | (FTU) | particles |
| — | Initial | 10.1 | 6.15 | 91.29 | 2.57 | 48.4 | 38.8 | 12.8 | 3.01 | Free from |
| | | | | | | | | | | particles |
| Shaking | 1 week | 10.1 | 6.18 | 91.74 | 2.08 | 48.0 | 38.7 | 13.3 | 2.60 | Free from |
| 5° C. | | | | | | | | | | particles |
| Shaking | 1 week | 10.3 | 5.84 | 91.55 | 2.61 | 46.6 | 38.7 | 14.6 | 2.71 | Free from |
| 25° C. | | | | | | | | | | particles |
| Freezing/ | — | 10.1 | 6.17 | 91.66 | 2.17 | 48.4 | 38.7 | 12.9 | 2.49 | Free from |
| Thawing | | | | | | | | | | particles |
| (5 cycles) | | | | | | | | | | |
| 5° C. | 5 weeks | 10.2 | 6.24 | 91.36 | 2.40 | 47.3 | 39.6 | 13.2 | 2.55 | Free from |
| | | | | | | | | | | particles |
| | 8 weeks | 10.2 | 6.18 | 91.05 | 2.77 | 47.2 | 37.7 | 13.1 | 2.85 | Free from |
| | | | | | | | | | | particles |
| 25° C. | 5 weeks | nd | 5.66 | 91.54 | 2.80 | 43.1 | 42.8 | 14.1 | 2.66 | Free from |
| | | | | | | | | | | particles |
| | 8 weeks | 10.1 | 6.20 | 90.58 | 3.22 | 39.4 | 46.0 | 14.6 | 4.39 | Free from |
| | | | | | | | | | | particles |
| 40° C. | 5 weeks | 10.1 | 5.26 | 89.75 | 4.98 | 21.1 | 63.6 | 15.4 | 2.75 | With many |
| | | | | | | | | | | particles |
| | 8 weeks | 10.1 | 4.64 | 88.71 | 6.65 | 14.4 | 73.7 | 12.2 | 3.16 | Essentially free |
| | | | | | | | | | | from particles |

Formulation C is a liquid formulation with the composition 10 mg/mL huMAb-CD44, 20 mM Citrate, 190 mM Sucrose, 20 mM L-Arginine, 0.02% Polysorbate 20, at pH 5.5

| | | Protein | Size Exclusion-HPLC | | | Ion Exchange-HPLC | | | | |
|---|---|---|---|---|---|---|---|---|---|---|
| Storage | Storage | concentration | HMW | Monomer | LMW | Main | Acidic | Basic | Turbidity | Visible |
| condition | time | (mg/mL) | (%) | (%) | (%) | Peak (%) | Peak (%) | Peak (%) | (FTU) | particles |
| — | Initial | 10.4 | 6.48 | 91.19 | 2.33 | 48.3 | 37.9 | 13.9 | 4.27 | Free from |
| | | | | | | | | | | particles |
| Shaking | 1 week | 10.4 | 6.53 | 91.21 | 2.26 | 48.0 | 38.2 | 13.8 | 4.30 | Free from |
| 5° C. | | | | | | | | | | particles |
| Shaking | 1 week | 10.5 | 6.36 | 90.99 | 2.65 | 46.9 | 38.7 | 14.4 | 4.00 | Free from |
| 25° C. | | | | | | | | | | particles |
| Freezing/ | — | 10.4 | 6.49 | 91.13 | 2.39 | 48.3 | 38.0 | 13.7 | 4.39 | Free from |
| Thawing | | | | | | | | | | particles |
| (5 cycles) | | | | | | | | | | |
| 5° C. | 5 weeks | 10.4 | 6.54 | 91.17 | 2.29 | 47.5 | 39.2 | 13.3 | 4.35 | Free from |
| | | | | | | | | | | particles |
| | 8 weeks | 10.4 | 6.52 | 90.99 | 2.49 | 48.0 | 38.6 | 13.5 | 5.01 | Free from |
| | | | | | | | | | | particles |
| 25° C. | 5 weeks | nd | 6.39 | 90.96 | 2.66 | 44.9 | 40.5 | 14.6 | 4.43 | Free from |
| | | | | | | | | | | particles |
| | 8 weeks | 10.4 | 6.36 | 90.52 | 3.12 | 42.9 | 42.2 | 14.9 | 5.02 | Free from |
| | | | | | | | | | | particles |
| 40° C. | 5 weeks | nd | 5.88 | 89.26 | 4.82 | 26.2 | 55.7 | 18.2 | 4.24 | Free from |
| | | | | | | | | | | particles |
| | 8 weeks | 10.0 | 5.61 | 87.97 | 6.42 | 17.8 | 65.1 | 17.1 | 4.89 | Free from |
| | | | | | | | | | | particles |

Preparation of Lyophilized Formulation

Solutions of approximately 10 mg/ml huMAb-CD44 were prepared as described above for liquid formulations. All formulations were sterile filtered through 0.22 μm filters and aseptically aliquoted into sterile 20 mL glass vials. The vials were partly closed with ETFE (Copolymer of ethylene and tetrafluoroethylene)-coated rubber stoppers suitable for the use in lyophilization processes and lyophilized using the freeze-drying cycle reported in Table 1.

TABLE 1
Freeze-drying Cycle

| | | Ramp | | Chamber |
|---|---|---|---|---|
| | Shelf temperature | Duration | Hold time | pressure |
| Step | (° C.) | (° C./min) | (min) | (mbar) |
| Pre-cooling | 5° C. | 0.0 | 60 | — |
| Freeze | −40° C. | 1.0 | 180 | — |
| Primary Drying | −25° C. | 0.5 | 4560 | 0.080 |
| Secondary Drying | +25° C. | 0.2 | 360 | 0.080 |

The product was first cooled from room temperature to approx 5° C. (pre-cooling), followed by a freezing step at −40° C. with a plate cooling rate of approximately 1° C./min, followed by a holding step at −40° C. for about 2 hours. The first drying step was performed at a plate temperature of approximately −25° C. and a chamber pressure of approximately 80 μbar for about 76 hours. Subsequently, the second drying step started with a temperature ramp of 0.2° C./min from −25° C. to 25° C., followed by a holding step at 25° C. for at least 5 hours at a chamber pressure of approximately 80 μbar.

Lyophilization was carried out in a Usifroid SMH-90 LN2 freeze-dryer (Usifroid, Maurepas, France) or a LyoStar II Freeze-dryer (FTS Systems, Stone Ridge, N.Y., USA). The freeze-dried samples were stored at different climate conditions (5° C., 25° C. and 40° C.) for different intervals of time. The lyophilized vials were reconstituted to a final volume of 5.3 mL with water for injection (WFI) yielding an isotonic formulation with an antibody concentration of approximately 10 mg/mL. The reconstitution time of the freeze-dried cakes was around 1 min. Analysis of the reconstituted samples was performed after a 24 hour incubation period of the reconstituted liquid sample at 25° C.

The samples were analyzed by the analytical methods 1) UV spectrophotometry, 2) Size Exclusion Chromatography (SEC), 3) by Ion exchange chromatography (IEC), 4) by turbidity of the solution and 5) for visible particles.

UV spectroscopy, used for determination of protein content, was performed on a Perkin Elmer λ 35 UV spectrophotometer in a wavelength range from 240 nm to 400 nm. Neat protein samples were diluted to approximately 0.5 mg/mL with the corresponding formulation buffer. The protein concentration was calculated according to equation 1.

$$Protein content = \frac{A ( 280 ) - A ( 320 ) \times dil . factor}{ɛ 〈 {cm}^{2} \text{/} mg 〉 \times d 〈 cm 〉} \qquad Equation 1$$

The UV light absorption at 280 nm was corrected for light scattering at 320 nm and multiplied with the dilution factor, which was determined from the weighed masses and densities of the neat sample and the dilution buffer. The numerator was divided by the product of the cuvette's path length d and the extinction coefficient E.

Size Exclusion Chromatography (SEC) was used to detect soluble high molecular weight species (aggregates) and low molecular weight hydrolysis products (LMW) in the formulations. The method was performed on a Waters Alliance 2695 HPLC instrument with a Waters W2487 Dual Absorbance Detector and equipped with a TosoHaas TSK-Gel G3000SWXL column. Intact monomer, aggregates and hydrolysis products were separated by an isocratic elution profile, using 0.2M K₂HPO₄/0.25M KCL, pH 7.0 as mobile phase, and were detected at a wavelength of 280 nm.

Ion Exchange Chromatography (IEC) was performed to detect chemical degradation products altering the net charge of huMAb-CD44 in the formulations. The method used a suitable HPLC instrument equipped with a UV detector (detection wavelength 280 nm) and a Dionex ProPac WCX-10, 4 mm×250 mm. 10 mM Sodium Phosphate pH 6.5 and 10 mM Sodium Phosphate including 1M Sodium Chloride, pH 6.5, were used as mobile phases A and B, respectively, with a flow rate of 1.0 mL/min.

For the determination of the turbidity, opalescence was measured in FTU (turbidity units) using a HACH 2100AN turbidimelter at room temperature.

### Compositions and Stability Data of Freeze-Dried huMAb-CD44 Drug Product Formulations According to this Invention

Formulation D is a lyophilized formulation with the composition 10 mg/mL huMAb-CD44, 20 mM L-Histidine, 240 mM Trehalose, 0.02% Polysorbate 20, at pH 6.0

| | | Protein | Size Exclusion-HPLC | | | Ion Exchange-HPLC | | | | |
|---|---|---|---|---|---|---|---|---|---|---|
| Storage | Storage | concentration | HMW | Monomer | LMW | Main | Acidic | Basic | Turbidity | Visible |
| condition | time | (mg/mL) | (%) | (%) | (%) | Peak (%) | Peak (%) | Peak (%) | (FTU) | particles |
| — | Initial | 9.7 | 6.16 | 91.42 | 2.41 | 48.6 | 38.3 | 13.1 | 2.80 | Essentially free |
| | | | | | | | | | | from particles |
| 5° C. | 5 weeks | 9.8 | 6.23 | 91.39 | 2.39 | 47.2 | 39.6 | 13.3 | 2.56 | Free from |
| | | | | | | | | | | particles |
| | 8 weeks | 9.9 | 6.21 | 91.10 | 2.68 | 47.5 | 39.2 | 13.3 | 2.84 | Essentially free |
| | | | | | | | | | | from particles |
| 25° C. | 5 weeks | nd | 6.23 | 91.38 | 2.40 | 46.6 | 39.0 | 14.5 | 2.67 | Essentially free |
| | | | | | | | | | | from particles |
| | 8 weeks | 9.8 | 6.21 | 91.08 | 2.70 | 46.9 | 39.2 | 13.9 | 3.91 | Free from |
| | | | | | | | | | | particles |
| 40° C. | 5 weeks | nd | 6.24 | 91.35 | 2.41 | 45.4 | 39.8 | 14.6 | 2.80 | Essentially free |
| | | | | | | | | | | from particles |
| | 8 weeks | 9.7 | 6.23 | 91.05 | 2.73 | 45.2 | 39.5 | 15.3 | 2.90 | Free from |
| | | | | | | | | | | particles |

Formulation E is a lyophilized formulation with the composition 10 mg/mL huMAb-CD44, 20 mM L-Histidine, 190 mM Sucrose, 20 mM L-Arginine, 0.02% Polysorbate 20, at pH 5.5

| | | Protein | Size Exclusion-HPLC | | | Ion Exchange-HPLC | | | | |
|---|---|---|---|---|---|---|---|---|---|---|
| Storage | Storage | concentration | HMW | Monomer | LMW | Main | Acidic | Basic | Turbidity | Visible |
| condition | time | (mg/mL) | (%) | (%) | (%) | Peak (%) | Peak (%) | Peak (%) | (FTU) | particles |
| — | Initial | 10.0 | 6.43 | 91.26 | 2.31 | 48.7 | 38.2 | 13.2 | 4.62 | Essentially free |
| | | | | | | | | | | from particles |
| 5° C. | 5 weeks | 10.1 | 6.42 | 91.26 | 2.33 | 47.8 | 38.7 | 13.5 | 439 | Free from |
| | | | | | | | | | | particles |
| | 8 weeks | 10.2 | 6.39 | 91.13 | 2.48 | 48.1 | 39.1 | 12.8 | 5.09 | Free from |
| | | | | | | | | | | particles |
| 25° C. | 5 weeks | nd | 6.42 | 91.25 | 2.32 | 47.1 | 39.5 | 13.4 | 4.27 | Free from |
| | | | | | | | | | | particles |
| | 8 weeks | 10.2 | 6.38 | 91.11 | 2.52 | 47.1 | 39.51 | 13.4 | 4.59 | Free from |
| | | | | | | | | | | particles |
| 40° C. | 5 weeks | nd | 6.44 | 91.24 | 2.32 | 45.0 | 41.2 | 13.9 | 4.28 | Free from |
| | | | | | | | | | | particles |
| | 8 weeks | 10.2 | 6.40 | 91.08 | 2.52 | 44.0 | 42.3 | 13.67 | 4.65 | Essentially free |
| | | | | | | | | | | from particles |

### Preparation of Liquid Formulations

For the preparation of the liquid formulations huMAb-CD44 was buffer-exchanged against a diafiltration buffer containing the anticipated buffer composition and where required, concentrated by diafiltration to an antibody concentration of approximately 40 mg/mL. After completion of the diafiltration operation, the excipients (e.g. sucrose) were added as stock solutions to the antibody solution. The surfactant was then added as a 50 to 200-fold stock solution. Finally the protein concentration was adjusted with a buffer to the final huMAb-CD44 concentration of approximately 25 mg/mL.

All formulations were sterile-filtered through 0.22 μm low protein binding filters and aseptically filled into sterile 6 mL glass vials closed with ETFE (Copolymer of ethylene and tetrafluoroethylene)-coated rubber stoppers and alucrimp caps. The fill volume was approximately 2.4 mL. These formulations were stored at different climate conditions (5° C., 25° C. and 40° C.) for different intervals of time and stressed by shaking (1 week at a shaking frequency of 200 min⁻¹ at 5° C. and 25° C.) and freeze-thaw stress methods. The samples were analyzed before and after applying the stress tests by the analytical methods 1) UV spectrophotometry, 2) Size Exclusion Chromatography (SEC), 3) by Ion exchange chromatography (IEC), 4) by turbidity of the solution and 5) for visible particles.

UV spectroscopy, used for determination of protein content, was performed on a Perkin Elmer λ 35 UV spectrophotometer in a wavelength range from 240 nm to 400 nm. Neat protein samples were diluted to approximately 0.5 mg/mL with the corresponding formulation buffer. The protein concentration was calculated according to equation 1.

$$Protein content = \frac{A ( 280 ) - A ( 320 ) \times dil . factor}{ɛ 〈 {cm}^{2} \text{/} mg 〉 \times d 〈 cm 〉} \qquad Equation 1$$

The UV light absorption at 280 nm was corrected for light scattering at 320 nm and multiplied with the dilution factor, which was determined from the weighed masses and densities of the neat sample and the dilution buffer. The numerator was divided by the product of the cuvette's path length d and the extinction coefficient E.

Size Exclusion Chromatography (SEC) was used to detect soluble high molecular weight species (aggregates) and low molecular weight hydrolysis products (LMW) in the formulations. The method was performed on a Waters Alliance 2695 HPLC instrument with a Waters W2487 Dual Absorbance Detector and equipped with a TosoHaas TSK-Gel G3000SWXL column. Intact monomer, aggregates and hydrolysis products were separated by an isocratic elution profile, using 0.2M K₂HPO₄/0.25M KCL, pH 7.0 as mobile phase, and were detected at a wavelength of 280 nm.

Ion Exchange Chromatography (IEC) was performed to detect chemical degradation products altering the net charge of huMAb-CD44 in the formulations. The method used a suitable HPLC instrument equipped with a UV detector (detection wavelength 280 nm) and a Dionex ProPac WCX-10, 4 mm×250 mm. 10 mM Sodium Phosphate pH 6.5 and 10 mM Sodium Phosphate including 1M Sodium Chloride, pH 6.5, were used as mobile phases A and B, respectively, with a flow rate of 1.0 mL/min.

For the determination of the turbidity, opalescence was measured in FTU (turbidity units) using a HACH 2100AN turbidimeter at room temperature.

Samples were analyzed for visible particles by using a Seidenader V90-T visual inspection instrument.
Formulation F is a liquid formulation with the composition 25 mg/mL huMAb-CD44, 20 mM L-histidine, 140 mM NaCl, 0.02% Polysorbate 20, at pH 5.5.

| | | Protein | Size Exclusion-HPLC | | | Ion Exchange-HPLC | | | | |
|---|---|---|---|---|---|---|---|---|---|---|
| Storage | Storage | concentration | HMW | Monomer | LMW | Main | Acidic | Basic | Turbidity | Visible |
| condition | time | (mg/mL) | (%) | (%) | (%) | Peak (%) | Peak (%) | Peak (%) | (FTU) | particles |
| — | Initial | 25.4 | 1.3 | 96.6 | 2.1 | 52.4 | 26.8 | 20.8 | 12.0 | Essentially free |
| | | | | | | | | | | from particles |
| Shaking | 1 week | 25.8 | 1.4 | 96.6 | 2.0 | 52.3 | 26.8 | 21.0 | 11.0 | Essentially free |
| 5° C. | | | | | | | | | | from particles |
| Shaking | 1 week | 25.7 | 1.4 | 96.3 | 2.2 | 50.9 | 27.9 | 21.2 | 11.4 | Free from |
| 25° C. | | | | | | | | | | particles |
| Freezing/ | — | 27.1 | 1.4 | 96.3 | 2.3 | 51.8 | 27.3 | 20.9 | 11.0 | Free from |
| Thawing | | | | | | | | | | particles |
| (5 cycles) | | | | | | | | | | |
| 5° C. | 4 weeks | 25.5 | 1.3 | 96.6 | 2.2 | 51.5 | 27.0 | 21.5 | 11.7 | Free from |
| | | | | | | | | | | particles |
| | 8 weeks | 25.1 | 1.4 | 96.6 | 2.0 | 51.9 | 27.2 | 20.9 | 12.4 | Free from |
| | | | | | | | | | | particles |
| 25° C. | 4 weeks | 26.0 | 1.8 | 95.6 | 2.6 | 48.0 | 30.1 | 21.9 | 12.3 | Essentially free |
| | | | | | | | | | | from particles |
| | 8 weeks | 25.6 | 2.0 | 95.3 | 2.7 | 48.8 | 29.2 | 22.0 | 13.5 | Essentially free |
| | | | | | | | | | | from particles |
| 40° C. | 4 weeks | 27.1 | 2.1 | 93.0 | 4.9 | 44.9 | 30.7 | 24.4 | 13.2 | Free from |
| | | | | | | | | | | particles |
| | 8 weeks | 25.4 | 2.9 | 89.9 | 7.2 | — | — | — | 13.9 | Free from |
| | | | | | | | | | | particles |

Formulation G is a liquid formulation with the composition 25 mg/mL huMAb-CD44, 20 mM L-histidine, 140 mM NaCl, 0.02% Polysorbate 20, at pH 6.0.

| | | Protein | Size Exclusion-HPLC | | | Ion Exchange-HPLC | | | | |
|---|---|---|---|---|---|---|---|---|---|---|
| Storage | Storage | concentration | HMW | Monomer | LMW | Main | Acidic | Basic | Turbidity | Visible |
| condition | time | (mg/mL) | (%) | (%) | (%) | Peak (%) | Peak (%) | Peak (%) | (FTU) | particles |
| — | Initial | 25.7 | 1.5 | 96.3 | 2.2 | 52.5 | 26.9 | 20.6 | 12.2 | Essentially free |
| | | | | | | | | | | from particles |
| Shaking | 1 week | 25.9 | 1.5 | 96.5 | 2.0 | 51.8 | 27.2 | 21.0 | 11.3 | Free from |
| 5° C. | | | | | | | | | | particles |
| Shaking | 1 week | 26.4 | 1.5 | 96.2 | 2.3 | 51.1 | 21.3 | 27.6 | 11.6 | Free from |
| 25° C. | | | | | | | | | | particles |
| Freezing/ | — | 26.5 | 1.5 | 96.5 | 2.0 | 51.8 | 20.9 | 27.3 | 12.0 | Free from |
| Thawing | | | | | | | | | | particles |
| (5 cycles) | | | | | | | | | | |
| 5° C. | 4 weeks | 25.9 | 1.4 | 96.5 | 2.1 | 51.4 | 21.4 | 27.1 | 11.0 | Free from |
| | | | | | | | | | | particles |
| | 8 weeks | 25.7 | 1.5 | 96.3 | 2.2 | 51.9 | 20.9 | 27.2 | 12.4 | Free from |
| | | | | | | | | | | particles |
| 25° C. | 4 weeks | 27.1 | 1.5 | 96.0 | 2.5 | 49.2 | 22.3 | 28.5 | 11.9 | Free from |
| | | | | | | | | | | particles |
| | 8 weeks | 25.6 | 1.7 | 95.6 | 2.6 | 47.7 | 22.7 | 29.6 | 13.6 | Free from |
| | | | | | | | | | | particles |
| 40° C. | 4 weeks | 27.1 | 1.9 | 94.1 | 4.1 | 41.5 | 26.8 | 31.7 | 13.0 | Free from |
| | | | | | | | | | | particles |
| | 8 weeks | 25.9 | 2.3 | 91.6 | 5.8 | — | — | — | 13.7 | Free from |
| | | | | | | | | | | particles |

Formulation H is a liquid formulation with the composition 25 mg/mL huMAb-CD44, 20 mM L-histidine, 140 mM NaCl, 0.02% Poloxamer 188, at pH 5.5

| | | Protein | Size Exclusion-HPLC | | | Ion Exchange-HPLC | | | | |
|---|---|---|---|---|---|---|---|---|---|---|
| Storage | Storage | concentration | HMW | Monomer | LMW | Main | Acidic | Basic | Turbidity | Visible |
| condition | time | (mg/mL) | (%) | (%) | (%) | Peak (%) | Peak (%) | Peak (%) | (FTU) | particles |
| — | Initial | 25.3 | 1.3 | 96.5 | 2.1 | 52.2 | 27.1 | 20.7 | 11.0 | Essentially free |
| | | | | | | | | | | from particles |
| Shaking | 1 week | 25.4 | 1.4 | 96.7 | 2.0 | 52.1 | 26.9 | 21.0 | 10.6 | Free from |
| 5° C. | | | | | | | | | | particles |
| Shaking | 1 week | 25.7 | 1.4 | 96.4 | 2.3 | 52.0 | 26.8 | 21.2 | 11.2 | Free from |
| 25° C. | | | | | | | | | | particles |
| Freezing/ | — | 26.9 | 1.4 | 96.6 | 2.0 | 52.3 | 26.8 | 20.9 | 11.1 | Free from |
| Thawing | | | | | | | | | | particles |
| (5 cycles) | | | | | | | | | | |
| 5° C. | 4 weeks | 25.4 | 1.3 | 96.5 | 2.2 | 51.7 | 26.9 | 21.4 | 11.1 | Free from |
| | | | | | | | | | | particles |
| | 8 weeks | 25.4 | 1.4 | 96.5 | 2.1 | 52.2 | 27.0 | 20.8 | 11.4 | Free from |
| | | | | | | | | | | particles |
| 25° C. | 4 weeks | 26.2 | 1.3 | 96.4 | 2.3 | 51.6 | 26.5 | 21.9 | 11.2 | Free from |
| | | | | | | | | | | particles |
| | 8 weeks | 25.3 | 1.4 | 96.1 | 2.5 | 51.4 | 26.4 | 22.3 | 11.5 | Free from |
| | | | | | | | | | | particles |
| 40° C. | 4 weeks | 26.3 | 1.4 | 94.5 | 4.1 | 47.8 | 27.2 | 25.0 | 11.4 | Free from |
| | | | | | | | | | | particles |
| | 8 weeks | 25.4 | 1.7 | 92.2 | 6.2 | — | — | — | 14.4 | Free from |
| | | | | | | | | | | particles |

Formulation I is a liquid formulation with the composition 25 mg/mL huMAb-CD44, 20 mM L-histidine, 140 mM NaCl, 0.02% Poloxamer 188, at pH 6.0

| | | Protein | Size Exclusion-HPLC | | | Ion Exchange-HPLC | | | | |
|---|---|---|---|---|---|---|---|---|---|---|
| Storage | Storage | concentration | HMW | Monomer | LMW | Main | Acidic | Basic | Turbidity | Visible |
| condition | time | (mg/mL) | (%) | (%) | (%) | Peak (%) | Peak (%) | Peak (%) | (FTU) | particles |
| — | Initial | 25.4 | 1.5 | 96.3 | 2.2 | 52.0 | 27.3 | 20.7 | 11.0 | Free from |
| | | | | | | | | | | particles |
| Shaking | 1 week | 25.6 | 1.5 | 96.5 | 2.0 | 53.0 | 27.1 | 13.1 | 11.3 | Essentially free |
| 5° C. | | | | | | | | | | from particles |
| Shaking | 1 week | 25.4 | 1.5 | 96.4 | 2.2 | 51.5 | 27.2 | 21.3 | 11.5 | Free from |
| 25° C. | | | | | | | | | | particles |
| Freezing/ | — | 26.6 | 1.5 | 96.2 | 2.3 | 51.9 | 27.1 | 21.0 | 11.2 | Free from |
| Thawing | | | | | | | | | | particles |
| (5 cycles) | | | | | | | | | | |
| 5° C. | 4 weeks | 26.0 | 1.4 | 96.4 | 2.2 | 51.5 | 27.1 | 21.4 | 11.3 | Free from |
| | | | | | | | | | | particles |
| | 8 weeks | 25.6 | 1.5 | 96.6 | 1.9 | 51.9 | 27.2 | 21.0 | 12.2 | Free from |
| | | | | | | | | | | particles |
| 25° C. | 4 weeks | 26.9 | 1.4 | 96.2 | 2.4 | 50.7 | 26.9 | 22.4 | 11.7 | Free from |
| | | | | | | | | | | particles |
| | 8 weeks | 25.5 | 1.5 | 96.1 | 2.4 | 49.9 | 26.8 | 23.3 | 12.5 | Free from |
| | | | | | | | | | | particles |
| 40° C. | 4 weeks | 26.9 | 1.4 | 94.6 | 3.7 | 43.0 | 29.1 | 27.9 | 12.5 | Free from |
| | | | | | | | | | | particles |
| | 8 weeks | 25.2 | 1.7 | 92.9 | 5.5 | — | — | — | 15.0 | Free from |
| | | | | | | | | | | particles |

Formulation J is a liquid formulation with the composition 25 mg/mL huMAb-CD44, 20 mM L-histidine, 240 mM Sucrose, 0.02% Poloxamer 188, at pH 5.5

| | | Protein | Size Exclusion-HPLC | | | Ion Exchange-HPLC | | | | |
|---|---|---|---|---|---|---|---|---|---|---|
| Storage | Storage | concentration | HMW | Monomer | LMW | Main | Acidic | Basic | Turbidity | Visible |
| condition | time | (mg/mL) | (%) | (%) | (%) | Peak (%) | Peak (%) | Peak (%) | (FTU) | particles |
| — | Initial | 24.9 | 1.2 | 96.5 | 2.3 | 56.2 | 27.1 | 20.3 | 5.1 | Free from |
| | | | | | | | | | | particles |
| Shaking | 1 week | 25.7 | 1.2 | 96.7 | 2.1 | 52.3 | 20.4 | 27.3 | 4.7 | Free from |
| 5° C. | | | | | | | | | | particles |
| Shaking | 1 week | 25.9 | 1.1 | 96.6 | 2.3 | 52.0 | 27.4 | 20.6 | 4.7 | Free from |
| 25° C. | | | | | | | | | | particles |
| Freezing/ | — | 26.7 | 1.2 | 96.5 | 2.3 | 52.3 | 27.2 | 20.6 | 5.4 | Free from |
| Thawing | | | | | | | | | | particles |
| (5 cycles) | | | | | | | | | | |
| 5° C. | 4 weeks | 25.7 | 1.2 | 96.6 | 2.3 | 51.7 | 27.1 | 21.2 | 4.6 | Free from |
| | | | | | | | | | | particles |
| | 8 weeks | 25.7 | 1.2 | 96.7 | 2.1 | 52.2 | 27.3 | 20.5 | 4.9 | Free from |
| | | | | | | | | | | particles |
| 25° C. | 4 weeks | 25.7 | 1.1 | 96.6 | 2.3 | 51.4 | 27.4 | 21.2 | 4.7 | Free from |
| | | | | | | | | | | particles |
| | 8 weeks | 25.7 | 1.1 | 96.4 | 2.6 | 51.3 | 27.6 | 21.2 | 5.2 | Free from |
| | | | | | | | | | | particles |
| 40° C. | 4 weeks | 25.9 | 1.0 | 95.2 | 3.9 | 45.5 | 32.5 | 22.0 | 4.8 | Free from |
| | | | | | | | | | | particles |
| | 8 weeks | 25.3 | 1.1 | 93.2 | 5.7 | — | — | — | 5.0 | Free from |
| | | | | | | | | | | particles |

Formulation K is a liquid formulation with the composition 25 mg/mL huMAb-CD44, 20 mM L-histidine, 240 mM Sucrose, 0.02% Poloxamer 188, at pH 6.0

| | | Protein | Size Exclusion-HPLC | | | Ion Exchange-HPLC | | | | |
|---|---|---|---|---|---|---|---|---|---|---|
| Storage | Storage | concentration | HMW | Monomer | LMW | Main | Acidic | Basic | Turbidity | Visible |
| condition | time | (mg/mL) | (%) | (%) | (%) | Peak (%) | Peak (%) | Peak (%) | (FTU) | particles |
| — | Initial | 25.1 | 1.3 | 96.6 | 2.2 | 51.2 | 28.1 | 20.0 | 4.3 | Free from |
| | | | | | | | | | | particles |
| Shaking | 1 week | 25.9 | 1.3 | 96.5 | 2.2 | 51.4 | 28.6 | 20.0 | 4.1 | Free from |
| 5° C. | | | | | | | | | | particles |
| Shaking | 1 week | 26.0 | 1.2 | 96.4 | 2.4 | 50.9 | 29.1 | 20.0 | 4.3 | Essentially |
| 25° C. | | | | | | | | | | free from |
| | | | | | | | | | | particles |
| Freezing/ | — | 27.7 | 1.3 | 96.4 | 2.4 | 51.6 | 28.3 | 20.1 | 3.9 | Free from |
| Thawing | | | | | | | | | | particles |
| (5 cycles) | | | | | | | | | | |
| 5° C. | 4 weeks | 27.1 | 1.2 | 95.5 | 2.3 | 51.5 | 27.8 | 20.7 | 4.3 | Free from |
| | | | | | | | | | | particles |
| | 8 weeks | 25.7 | 1.3 | 96.5 | 2.2 | 51.7 | 28.2 | 20.1 | 4.2 | Free from |
| | | | | | | | | | | particles |
| 25° C. | 4 weeks | 27.4 | 1.1 | 96.4 | 2.4 | 49.7 | 30.0 | 20.3 | 4.1 | Free from |
| | | | | | | | | | | particles |
| | 8 weeks | 25.5 | 1.2 | 96.3 | 2.6 | 47.9 | 32.4 | 19.7 | 4.4 | Free from |
| | | | | | | | | | | particles |
| 40° C. | 4 weeks | 27.7 | 1.2 | 95.0 | 3.9 | 35.1 | 47.2 | 17.7 | 4.4 | Free from |
| | | | | | | | | | | particles |
| | 8 weeks | 25.7 | 1.4 | 93.0 | 5.6 | — | — | — | 7.7 | Free from |
| | | | | | | | | | | particles |

1. A formulation comprising:
about 5 to about 30 mg/mL of a CD44 antibody,
about 5 to about 50 mM of a buffer,
at a pH from about 4.5 to about 7.0.

2-23. (canceled)

24. The formulations according to claim 1, wherein the CD44 antibody comprises one or more of the CDRs selected from the group of SEQ ID NO:3, SEQ ID NO:4, SEQ ID NO:5, SEQ ID NO:6, SEQ ID NO:7, or SEQ ID NO:8.

25. The formulation according to claim 24, wherein the antibody is humanized.

26. The formulation according to claim 24, wherein the antibody comprises SEQ ID NO:9.

27. The formulation according to claim 24, wherein the antibody comprises SEQ ID NO:10.

28. The formulation according to claim 24, wherein the antibody further comprises SEQ ID NO:11.

29. The formulation according to claim 24, wherein the formulation selected from the group consisting of a liquid formulation comprising:
a)—10 mg/mL of the CD44 antibody,
20 mM L-histidine,
150 mM NaCl,
0.01% polysorbate 80,
at a pH of 6.0;
b)—10 mg/mL of the CD44 antibody,
20 mM L-histidine,
240 mM trehalose,
0.02% polysorbate 20,
at a pH of 6.0;
c)—10 mg/mL of the CD44 antibody,
20 mM citrate,
190 mM sucrose,
20 mM L-arginine,
0.02% polysorbate 20,
at pH 5.5;
d)—25 mg/mL of the CD44 antibody,
20 mM L-histidine,
140 mM NaCl,
0.02% Poloxamer 188,
at pH of 5.5;
e)—25 mg/mL of the CD44 antibody,
20 mM L-histidine,
140 mM NaCl,
0.02% Polysorbate 20,
at a pH of 5.5;
f)—25 mg/mL of the CD44 antibody,
20 mM L-histidine,
140 mM NaCl,
0.02% Polysorbate 20,
at a pH of 6.0;
g)—25 mg/mL of the CD44 antibody,
20 mM L-histidine,
140 mM NaCl,
0.02% Poloxamer 188,
at a pH of 6.0;
h)—25 mg/mL of the CD44 antibody,
20 mM L-histidine,
240 mM Sucrose,
0.02% Poloxamer 188,
at a pH of 5.5;
and
i)—25 mg/mL of the CD44 antibody,
20 mM L-histidine,
240 mM Sucrose,
0.02% Poloxamer 188,
at a pH of 6.0.

30. The formulation according to claim 24, wherein the formulation is selected from the group consisting of a lyophilized formulation comprising:
a)—10 mg/mL of the CD44 antibody,
20 mM L-histidine,
240 mM trehalose,
0.02% polysorbate 20,
at pH 6.0
and
b)—10 mg/mL of the CD44 antibody,
20 mM L-histidine,
190 mM sucrose,
20 mM L-arginine,
0.02% polysorbate 20,
at a pH of 5.5.

31. The formulation according to claim 24, comprising:
10 mg/mL of the CD44 antibody,
150 mM NaCl,
20 mM of a L-histidine,
0.01% of polysorbate 80,
at a pH of 6.

32. The formulation according to claim 24, comprising:
25 mg/mL of the CD44 antibody,
20 mM L-histidine,
140 mM NaCl,
0.02% Poloxamer 188,
at a pH of 5.5.

33-35. (canceled)

36. A method of treating a patient suffering from a disease modulated by the CD44 receptor, comprising administering to the patient the formulation according to claim 1.

37. The method of claim 36, wherein the disease is selected from the group consisting of breast cancer, hepatocellular carcinoma, acute myeloid leukemia and squameous cell carcinoma.


SEQUENCE LISTING (12 sequences)

SEQ ID NO: 1 - protein, 124 aa, Homo sapiens
EVKLLESGGGLVQPGGSLKLSCATSGFDFSRYWMSWVRQAPGKGLEWIGEVNPDSTSINYTPSLKDQFIISRDNAKNTLDLQMSKVSSEDTALYYCTRPNYYGSRYHYYAMDYWGQGTSVTVSS

SEQ ID NO: 2 - protein, 107 aa, Homo sapiens
DIQMTQTTSSLSVSLGDRVTINCRASQDINNYLNWYQQKPDGTVKLLIYYTSRLHSGVPSRFSGSGSGTDFSLTISNLEKEDVATYFCQQGSTLPFTFGSGTKLEIK

SEQ ID NO: 3 - protein, 5 aa, Homo sapiens
RYWMS

SEQ ID NO: 4 - protein, 17 aa, Homo sapiens
EVNPDSTSINYTPSLKD

SEQ ID NO: 5 - protein, 15 aa, Homo sapiens
PNYYGSRYHYYAMDY

SEQ ID NO: 6 - protein, 11 aa, Homo sapiens
RASQDINNYLN

SEQ ID NO: 7 - protein, 7 aa, Homo sapiens
YTSRLHS

SEQ ID NO: 8 - protein, 9 aa, Homo sapiens
QQGSTLPFT

SEQ ID NO: 9 - protein, 124 aa, Homo sapiens
EVQLVESGGGLVQPGGSLRLSCAASGFDFSRYWMSWVRQAPGKGLVWVGEVNPDSTSINYTPSLKDRFTISRDNAKNTLYLQMNSLRAEDTAVYYCTRPNYYGSRYHYYAMDYWGQGTLVTVSS

SEQ ID NO: 10 - protein, 124 aa, Homo sapiens
EVQLVESGGGLVQPGGSLRLSCATSGFDFSRYWMSWVRQAPGKGLVWIGEVNPDSTSINYTPSLKDQFTISRDNAKNTLYLQMNSLRAEDTAVYYCTRPNYYGSRYHYYAMDYWGQGTLVTVSS

SEQ ID NO: 11 - protein, 107 aa, Homo sapiens
DIQMTQSPSSLSASVGDRVTITCRASQDINNYLNWYQQKPGKAPKLLIYYTSRLHSGVPSRFSGSGSGTDFTFTISSLQPEDIATYYCQQGSTLPFTFGQGTKLEIK

SEQ ID NO: 12 - protein, 11 aa, Homo sapiens
AFDGPITITIV